SEQUENCE LISTING (17 sequences)

SEQ ID NO: 1 - protein, 452 aa, synthetic construct
QVQLQESGPGLVKPSETLSLTCAVSGYSISSGYYWGWIRQPPGKGLEWIGTIYHSGSTYYNPSLKSRVTISVDTSKNQFSLKLSSVTAADTAVYYCARQGSIKQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, synthetic construct
DIVMTQSPLSLPVTPGEPASISCRSSQSLLRSTGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 9 aa, synthetic construct
YTFTKYYMS

SEQ ID NO: 4 - protein, 17 aa, synthetic construct
IINPIGGSTSYAQKFQG

SEQ ID NO: 5 - protein, 22 aa, synthetic construct
ARDPSGIALAGPASRGYQGMDV

SEQ ID NO: 6 - protein, 11 aa, synthetic construct
RASQSVSSNLA

SEQ ID NO: 7 - protein, 7 aa, synthetic construct
GASTRAT

SEQ ID NO: 8 - protein, 9 aa, synthetic construct
QQARLGPWT

SEQ ID NO: 9 - RNA, 20 nt, synthetic construct
  source 1..20: mol_type = other RNA
aggtggtgttaacagcagag

SEQ ID NO: 10 - RNA, 20 nt, synthetic construct
  source 1..20: mol_type = other RNA
aatgttccaatgccccactc

SEQ ID NO: 11 - RNA, 21 nt, synthetic construct
  source 1..21: mol_type = other RNA
  variation 20..21: note = deoxyribonulceotide
ctctgctgttaacaccacctt

SEQ ID NO: 12 - RNA, 20 nt, synthetic construct
  source 1..20: mol_type = other RNA
tttacaacctatgcaaatgg

SEQ ID NO: 13 - RNA, 25 nt, synthetic construct
  source 1..25: mol_type = other RNA
ccaagtcaaattctgtccctattat

SEQ ID NO: 14 - RNA, 25 nt, synthetic construct
  source 1..25: mol_type = other RNA
gagaactctggaaaggtgatactaa

SEQ ID NO: 15 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQARLGPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 459 aa, synthetic construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 458 aa, synthetic construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0366023 A1
(43) Pub. Date: Nov. 19, 2026

(54) SORTILIN INHIBITORS FOR TREATMENT OF PATIENTS WITH FUNCTIONAL NEUROENDOCRINE TUMORS

(71) Applicant: Bertram WIEDENMANN, Berlin (BE)

(72) Inventors: Bertram WIEDENMANN, Berlin (DE); Michael SIGAL, Berlin (DE); Felix BOLDUAN, Berlin (DE)

(21) Appl. No.: 19/487,498

(22) PCT Filed: May 10, 2024
(86) PCT No.: PCT/EP2024/062984

§ 371 Date: Nov. 25, 2025

(30) Foreign Application Priority Data
May 26, 2023 (EP) 23175836.8

(51) Int. Cl. C12N 15/113; A61K 31/44; A61K 31/7105; C07K 16/28

(52) U.S. Cl. CPC C12N 15/113; A61K 31/44; A61K 31/7105; C07K 16/28; C07K 2317/56; C12N 2310/11; C12N 2310/14

(57) ABSTRACT

The present invention relates to the field of anti-cancer therapies, more specifically to therapies with the aim of improving the health of patients suffering from functional neuroendocrine tumors (NETs) or functional neuroendocrine neoplasms (NENs). In particular, the invention discloses the use of sortilin inhibitors for treating a condition associated with the release of an endocrine and/or a paracrine factor such as serotonin by a functional neuroendocrine neoplasm in a subject, e.g., carcinoid syndrome, carcinoid heart disease, and/or carcinoid crisis. The sortilin inhibitor can be provided in a pharmaceutical composition and may be a small molecule inhibitor having a molecular weight of 800 daltons or less, an anti-sortilin antibody, an inhibitory nucleic acid, a proteolysis-targeting chimera, a peptide-based inhibitor or a combination thereof.

The present invention relates to the field of anti-cancer therapies, more specifically to therapies with the aim of improving the health of patients suffering from functional neuroendocrine tumors (NETs) or functional neuroendocrine neoplasms (NENs). In particular, the invention discloses the use of sortilin inhibitors for treating a condition associated with the release of an endocrine and/or a paracrine factor such as serotonin by a functional neuroendocrine neoplasm in a subject, e.g., carcinoid syndrome, carcinoid heart disease, and/or carcinoid crisis. The sortilin inhibitor can be provided in a pharmaceutical composition and may be a small molecule inhibitor having a molecular weight of 800 daltons or less, an anti-sortilin antibody, an inhibitory nucleic acid, a proteolysis-targeting chimera, a peptide-based inhibitor or a combination thereof.

Neuroendocrine tumors (NET) are rare tumors arising from hormone-producing cells that have an incidence of about 2-5 per 100,000 population. During the last decades, the age-adjusted incidence rate increased significantly from 1.09 (1973) to 6.98 per 100,000 (2012) (Dasari et al., 2017). The cause of the development of NETs is largely unknown, but in some cases, it can be attributed to genetic alterations in neuroendocrine cells. NETs comprise cells exerting endocrine as well as neural cell characteristics. While they can occur in every organ, they are particularly predominant in the gastroenteropancreatic system (Rindi et al., 2020).

Approximately ⅓ of all NETs are classified as “functional”, meaning they are characterized by their ability to produce and release excessive amounts of bioactive substances, especially hormones and biogenic amines such as serotonin. Once these substances are distributed throughout the body via the blood circulation, affected patients frequently develop various hormone-induced symptoms and syndromes that further limit their well-being and overall health in addition to the immediate effects caused by the tumor itself. One of the most common hormone mediated syndromes that occur secondary to neuroendocrine tumors is known as carcinoid syndrome. Carcinoid syndrome occurs mainly due to an over-secretion of serotonin, which can be observed in 32% of all small intestinal NETs (Halperin et al., 2017). Patients suffering from this syndrome develop various symptoms including diarrhea as well as cardiac complications and often exhibit a significantly shorter overall survival than patients affected by non-functional NETs that do not secrete bioactive substances. (Halperin et al., 2017, Joish et al., 2019).

Treatment of carcinoid syndrome is currently limited to controlling the proliferation of the primary tumor and containment of the various symptoms. The latter is predominantly achieved by administering somatostatin analogues (SSA) to affected patients. These analogues block receptor-mediated hormone release by NETs and also exert a relatively weak growth inhibitory effect. However, given that, after all, patients suffering from this tumor-associated disease can generally live for many years, it is common for SSAs to lose their effect with increasing treatment duration. In addition, treatment is further hampered due to an increase of the hormone activity with the duration of the disease.

Peptide directed radiotherapy (PRRT) is another alternative treatment for patients who failed somatostatin analogue therapy. This method uses radioactive somatostatin analogues to target the tumor directly. However, studies that test PRRT-approaches have so far been limited only to short periods of time (Ito et al, 2018) As of today, it is unknown what distinguishes a functional from a non-functional NET on a molecular level. This lack of knowledge has so far prohibited more targeted therapeutic options for the treatment of carcinoid syndrome.

Thus, there is a significant need for further treatment options to more efficiently and durably treat the various hormone-induced syndromes and symptoms associated with functional NETs.

This problem is solved by the present invention, in particular by the subject matter of the claims.

The present invention provides a sortilin inhibitor for use in treating a condition associated with the release of an endocrine and/or a paracrine factor by a functional neuroendocrine tumor in a subject.

Also disclosed herein is a method of treating a condition associated with the release of an endocrine and/or a paracrine factor by a functional neuroendocrine tumor in a subject, wherein the method comprises a step of administering to the subject a therapeutically effective amount of a sortilin inhibitor.

Sortilin, also known as neurotensin receptor 3 or SORT1, is a member of the VPS10P domain sorting receptor family, a group of transmembrane receptors which are involved in sorting and intracellular trafficking of a broad range of ligands (Malik et al., 2020). Sortilin furthermore serves as a co-receptor for the precursor of nerve growth factor (pro-NGF) and the 75-kDa neurotrophin receptor (p75NTR). It has been shown that sortilin is expressed in many healthy as well as in many cancer cells like breast cancer or lung cancer (Roselli et al., 2015; Al-Akhrass et al., 2017; Gao et al., 2018). Expression was also found in NETs and was attributed to cell migration and adhesion (Kim et al., 2018). However, only a fraction of the stained tissue samples were positive for sortilin expression (Kim et al., 2018).

NETs and their cells are characterized by the cell type-specific presence of so-called neurosecretory vesicles. Synaptophysin has been shown in the past to be an essential molecular component of these neurosecretory vesicles and can be used as a central marker molecule for the immunohistochemical diagnosis of NETs. Release of the neurosecretory vesicles leads to the dispersal of the endocrine and paracrine factors that cause NET-specific symptoms and syndromes. The present inventors thus addressed the molecular mechanisms that control the release of neurosecretory vesicles. Surprisingly, the inventors discovered sortilin to be a central regulatory molecule of the neurosecretory release of the endocrine and/or paracrine factors. Correspondingly, the inventors were able to show that inhibition of sortilin using a suitable sortilin inhibitor effectively prevents the release of these factors by the cells of NETs.

Treating a condition is herein to be understood to comprise a curative medical therapy of a subject with the intent to cure, ameliorate or stabilize a condition. The term “condition” refers to a disease, a syndrome, a disorder or a particular physiological state of an organism that is either manifested by distinct symptoms or can be diagnosed by using established markers capable of recognizing said state. In some embodiments, treating a condition is intended to mean that the progression of the condition is to be slowed, stopped or, preferably, reversed to allow for a perceivable improvement of the subject's well-being and health.

In one embodiment, the sortilin inhibitor may also be for use in preventing a condition associated with the release of an endocrine and/or a paracrine factor by a functional neuroendocrine tumor in a subject. Preventing a condition refers to precluding said condition from happening in the first place. Preventing a condition thus also explicitly includes the prophylactic treatment of a subject.

The subject typically is a mammal, e.g., a human, non-human primate, horse, pig, rabbit, dog, sheep, goat, cow, camel, cat, guinea pig, rat or mouse. In a preferred embodiment, the subject is a human.

In the context of the present invention, the terms neuroendocrine neoplasm (NEN) and neuroendocrine tumor (NET) are used synonymously. As the name implies, NETs originate from specialized cells of the body's neuroendocrine system that exhibit traits of both hormone-producing endocrine cells and nerve cells. NETs most commonly develop in the gastrointestinal tract, as about 40% of all NETs arise in the colon and small intestine and 4% in the appendix. However, many NETs also develop in the lung (approximately 30%) and pancreas (approximately 7%). Beside local symptoms, NETs can secrete various endocrine and/or paracrine factors such as, e.g., hormones and other vasoactive substances such as biogenic amines. The ability to secrete endocrine and/or paracrine factors divides NETs in “functional” and “non-functional”. Accordingly, in the context of the present invention, the term “functional NET” (often also referred to as “clinically functioning” or “symptomatic” NET) is herein understood to refer to a NET that generates and secretes endocrine and/or paracrine factors, e.g., vasoactive biogenic amines or hormones.

The functional NET preferably is a gastrointestinal NET (i.e., a colon NET, a small intestine NET or an appendix NET). The NET may however also be a lung NET or a pancreas NET.

In rarer instances, NETs may also develop in or on adrenal glands and are referred to as pheochromocytoma and paraganglioma. Therefore, in some embodiments, the NET may also be a pheochromocytoma or a paraganglioma.

In the context of the invention, the term “condition associated with the release of an endocrine and/or paracrine factor by a functional neuroendocrine tumor” typically refers to a condition that is associated with the NET but is mediated by one or more endocrine and/or paracrine factor, e.g., a hormones and/or vasoactive substance, secreted by said NET.

An endocrine factor is a signaling molecule of the endocrine system that is released by an internal gland. It exerts its effects far from its site of production, i.e., it is transported via the blood circulation to a different organ, where it can regulate physiology and/or behaviour.

Endocrine factors are more commonly referred to as hormones. They can comprise various types of substances, including eicosanoids such as, e.g., prostaglandins, steroids, amino acid derivatives as wells as proteins and peptides.

In contrast to endocrine factors, paracrine factors are not distributed via the blood circulation to distant targets but diffuse over relatively short distances. Thus, they exert local effects in the environment in which they are produced.

Many of the substances secreted by functional NETs may exert both endocrine as well as paracrine effects. Therefore, the distinction between endocrine and paracrine factors in the context of the invention need not necessarily refer to two different groups of biologically active substances, but may just as well be used to ascribe different (i.e., endocrine and paracrine) functions to one and the same factor. Preferably, the factor has at least endocrine functions.

The endocrine and/or paracrine factor released by the functional neuroendocrinic tumor and contributing to the condition to be treated preferably is a biogenic amine such as serotonin, histamine or a (poly-)peptide such as bradykinin, tachykinin (substance P), prostaglandin and/or kallikrein, preferably serotonin.

However, the endocrine and/or paracrine factor secreted by the NET may also be, e.g., adrenaline, gastrin, insulin, glucagon, somatostatin, corticotropin-releasing hormone, adrenocorticotropin, somatoliberin, somatotropin, parathyroid hormone-related protein, calcitonin, calcitonin gene-related peptide, vasoactive intestinal polypeptide, chromogranin A, katacalcin, neuropeptide Y, bombesin/gastrin releasing peptide (GRP), alpha-human chorionic gonadotropin (aHCG), thyroid stimulating hormone-like peptide, cholecystokinin, adrenomedullin, vascular endothelial growth factor (VEGF) and any combination thereof.

In a preferred embodiment, the condition associated with the release of an endocrine and/or paracrine factor by a functional neuroendocrine tumor is carcinoid syndrome. Carcinoid syndrome occurs in about 10% of all neuroendocrine tumors and is characterized by various symptoms, including skin flushing, in particular at the head and the upper part of the thorax, facial skin lesions, diarrhea, oftentimes associated with abdominal cramping and pain, bronchoconstriction as well as a rapid heartbeat. Further symptoms include malabsorption, fatigue, muscle loss and cognitive impairment. Normally, the liver is capable of neutralizing aberrantly secreted hormones and other biologically active substance before they have a chance to travel through the body and cause the symptoms associated with carcinoid syndrome. However, this protective first pass metabolism is often bypassed once NENs metastasize to the liver itself. Moreover, carcinoid syndrome may also arise in the absence of liver metastases due to neuroendocrine tumors in the bronchi, as their vasoactive products may reach the blood circulation before passing through the liver. Many of the symptoms associated with carcinoid syndrome primarily develop due to an altered tryptophan metabolism in the neuroendocrine tumor leading to the release of serotonin into the blood stream. However, additional vasoactive substances are thought to contribute to the manifestations of carcinoid syndrome, including histamine, tachykinins, kallikrein and prostaglandins.

In some embodiments, the condition associated with the release of endocrine and/or paracrine factors by a functional neuroendocrine neoplasm may also be carcinoid crisis. Carcinoid crisis refers to an extreme exacerbation of carcinoid syndrome due to prolonged and excessive release of endocrine and/or paracrine factors, in particular serotonin, by the NET. Carcinoid crisis manifests itself by symptoms including profound flushing, widely and rapidly fluctuating blood pressure, arrythmia, tachycardia, and bronchospasm. It may also occur in patients in which no carcinoid syndrome has been diagnosed before.

In further embodiments, the condition associated with the release of an endocrine and/or paracrine factor by a functional neuroendocrine neoplasm may be carcinoid heart disease. Carcinoid heart disease is the result of valvular damage related to the vasoactive factors released by the neuroendocrine tumor affecting, in particular, the tricuspid and the pulmonic valves. In many instances tissue injury around the heart valves is followed by plaque and fibrosis formation. Carcinoid heart disease typically develops as a consequence of prolonged carcinoid syndrome.

In some instances, the term “condition associated with the release of an endocrine and/or paracrine factor by a functional neuroendocrine tumor” may also refer to merely one of the herein described symptoms of carcinoid syndrome, carcinoid crisis or carcinoid heart disease. For instance, the condition may be any symptom selected from skin flushing, facial skin lesions, diarrhea, abdominal cramping and pain, bronchoconstriction, rapid heartbeat, malabsorption, fatigue, muscle loss, cognitive impairment, widely and rapidly fluctuating blood pressure, arrythmia, tachycardia, bronchospasm as well as tissue injury around the heart valves followed by plaque and fibrosis formation.

The excessive secretion of serotonin by NETs can further cause a depletion of tryptophan leading to niacin deficiency, and thus pellagra in the patient, which increases the risk of developing dermatitis, dementia, and diarrhea.

Many other hormones can be secreted by NETs, most commonly growth hormone (somatotropin) that can cause acromegaly, or cortisol, that can cause Cushing's syndrome. Excessive secretion of insulin results in low blood sugar levels, whereas uncontrolled release of glucagon is associated with the development of skin rashes and a disturbed sugar metabolism. Release of vasoactive intestinal polypeptide may cause diarrhea and increased urination (Verner-Morrison syndrome) and excessive secretion of gastrin increases the acidity in the stomach and small intestine and thus may cause heartburn, diarrhea and gastrointestinal ulcers (Zollinger-Ellison syndrome).

In further embodiments, the condition associated with the release of an endocrine and/or paracrine factor by a functional neuroendocrine neoplasm may thus also be niacin deficiency, pellagra, dermatitis, dementia, acromegaly, Cushing's syndrome, low blood sugar, skin rashes, disturbed sugar metabolism, Verner-Morrison syndrome or Zollinger-Ellison syndrome.

### Inhibitors

In the context of the invention, an inhibitor is understood as any compound, substance or composition capable of inhibiting a biological process or a chemical reaction. Thus, the term “sortilin inhibitor” refers to any compound, substance or composition (such as an antibody, protein, nucleic acid or other molecule) that interferes with, blocks, or otherwise attenuates either the expression and/or stability of sortilin itself or the interaction of sortilin with at least one of its ligands, e.g., with pro-neurotrophin. In the latter case, the sortilin inhibitor may for instance be a sortilin antagonist that competes with a naturally occurring sortilin ligand for binding to sortilin. For example, the sortilin inhibitor of the present invention may prevent the protein-protein interaction between a sortilin protein and its ligand pro-neurotrophin, thereby preventing the formation of the protein complex usually formed between sortilin, pro-neurotrophin and the p75NTR receptor, or resulting in the formation of a low affinity complex, which is biologically less active or inactive or has minimal activity.

Thus, in some embodiments, the sortilin inhibitor may completely prevent the interaction between sortilin and its ligands, or may do so only partially, in which case the complex between sortilin and its ligands may still form but may have reduced biological potency. The sortilin inhibitor may also interfere with, block or otherwise attenuate sortilin function.

The sortilin inhibitor may be a small molecule drug having a molecular weight of 800 daltons or less, an anti-sortilin antibody, an inhibitory nucleic acid or a peptide-based inhibitor.

In a first embodiment of the invention, the sortilin inhibitor is a small molecule drug. In the context of the present invention, the term “small molecule” refers to an organic compound having a molecular weight of less than 800 Da. The low molecular weight of small molecule drugs enables rapid diffusion of the compounds across cell membranes and increases their oral bioavailability.

Preferably, the small molecule inhibitor of sortilin is 2-[[(6-methyl-2-pyridinyl)amino]carbonyl]-5-(trifluoromethyl)-benzoic acid, which is better known under the name AF38469. AF38469 was first described by Schroder et al, in 2014 and is orally bioavailable and highly selective for sortilin over several of its naturally occurring ligands, including, e.g., neurotensin-1. As demonstrated in the Examples below, the inventors successfully used AF38469 as a sortilin inhibitor to suppress secretion of serotonin by NEN cells.

Alternatively, the small molecule sortilin inhibitor may be (2S)-2-{[(7-hydroxy-4-methyl-2-oxo-2H-chromen-8-yl)methyl]amino}-4-methylpentanoic acid (commonly referred to as AF40431), which was the first sortilin inhibitor ever identified, but exhibits comparably poor solubility and membrane permeability.

In another embodiment, the small molecule inhibitor of sortilin may also be 1-[2-(2-tert-butyl-5-methylphenoxy)-ethyl]-3-methylpiperidine (MPEP), as previously described in Lee et al., 2014. Unlike e.g., AF38469, which interferes with the interactions of sortilin with its ligands, MPEP appears to inhibit sortilin by increasing its degradation, thereby suppressing sortilin protein levels.

In recent years, numerous other small molecule inhibitors of sortilin have been developed and published. For instance, the small molecule sortilin inhibitor may also be any of the compounds disclosed in WO2021/186054 A1, which is hereby incorporated in its entirety by reference. The sortilin inhibitor may thus be a sortilin antagonist of Formula 1:

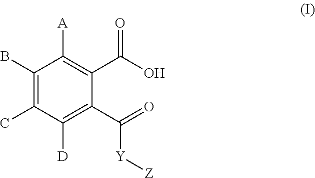

or a pharmaceutically acceptable salt, solvate, hydrate, geometrical isomer, tautomer, optical isomer, N-oxide, and/or prodrug thereof, wherein
Y is selected from the group consisting of —O—, —NR⁶—, and —S—;
Z is selected from the group consisting of optionally substituted C₁-C₆ alkyl, optionally substituted C₆-C₁ aryl, and optionally substituted C₁-C₉ heteroaryl,
wherein C₆-C₁₀ aryl and C₁-C₉ heteroaryl are optionally substituted with one or more substituents independently selected from the group consisting of —OR¹⁵, halo, and C₁-C₄ alkyl, wherein R¹⁵ is H, C₁-C₃ alkyl, or C₄ alkyl,
and/or wherein an alkylene group is attached to two adjacent atoms of the C₆-C₁₀ aryl or C₁-C₉ heteroaryl group to form a 5- or 6-membered partially saturated or saturated ring, optionally wherein the alkylene group is substituted with one or more halo atoms;
A, B, C and D are each independently selected from the group consisting of H, optionally substituted C₁-C₆ alkyl, halo, NO₂, optionally substituted C₆-C₁₀ aryl, optionally substituted C₁-C₉ heteroaryl, —OR⁶, NR⁷R⁸, —SR⁹, —C(O)OR¹⁰, —C(O)NR¹¹R¹², —C(O)SR¹³, C(O₂)SR¹⁴; R¹, R⁴, R⁵, R⁶, R⁹, R¹⁰, R¹³, and R¹⁴ are each independently selected from the group consisting of H and C₁-C₆ alkyl group, wherein the C₁-C₆ alkyl is optionally substituted with one or more halo atoms; and
R², R³, R⁷, R⁸, R¹¹, and R¹² are each independently selected from the group consisting of H and C₁-C₆ alkyl, wherein the C₁-C₆ alkyl is optionally substituted with one or more halo atoms, or R² and R³, R⁷ and R⁸, and/or R¹¹ and R¹² can be taken together with the nitrogen atom to which they are attached to from a 5- or 6-membered heterocycle, optionally substituted with one or more halo atoms.

In particular, the small molecule sortilin inhibitor may be any of 2-[(6-methylpyridin-2-yl)carbamoyl]benzoic acid, 2-methyl-6-[(6-methylpyridin-2-yl)carbamoyl]benzoic acid, 5-bromo-2-[(6-methylpyridin-2-yl)carbamoyl]benzoic acid, 5-chloro-2-[(6-methylpyridin-2-yl)carbamoyl]benzoic acid, 5-methyl-2-[(6-methylpyridin-2-yl)carbamoyl]benzoic acid, 2-[(6-methylpyridin-2-yl)carbamoyl]-5-(propan-2-yl)benzoic acid, 2-[(6-methylpyridin-2-yl)carbamoyl]-5-(trifluoromethyl)benzoic acid, 2-[(6-methylpyridin-2-yl)carbamoyl]-5-nitrobenzoic acid, 4-[(6-methylpyridin-2-yl)carbamoyl]-[1,1′-biphenyl]-3-carboxylic acid, 4-bromo-2-[(6-methylpyridin-2-yl)carbamoyl]benzoic acid, 4-chloro-2-[(6-methylpyridin-2-yl)carbamoyl]benzoic acid, 4-methyl-2-[(6-methylpyridin-2-yl)carbamoyl]benzoic acid, 2-[(6-methylpyridin-2-yl)carbamoyl]-4-(trifluoromethyl)benzoic acid, 4,5-dichloro-2-[(6-methylpyridin-2-yl)carbamoyl]benzoic acid, 4,5-dimethyl-2-[(6-methylpyridin-2-yl)carbamoyl]benzoic acid; 3-methyl-2-[(6-methylpyridin-2-yl)carbamoyl]benzoic acid, 5-bromo-2-[(butan-2-yl)carbamoyl]benzoic acid, 5-bromo-2-[(propan-2-yl)carbamoyl]benzoic acid, 5-bromo-2′-(phenylcarbamoyl)benzoic acid, 5-bromo-2-[(3-methylphenyl)carbamoyl]benzoic acid, 5-bromo-2-[(pyridin-2-yl)carbamoyl]benzoic acid, 5-bromo-2-[(6-chloropyridin-2-yl)carbamoyl]benzoic acid, 5-bromo-2-[(4-methylpyridin-2-yl)carbamoyl]benzoic acid, 5-bromo-2-[(3-methylpyridin-2-yl)carbamoyl]benzoic acid, 5-bromo-2-[(6-methylpyridin-3-yl)carbamoyl]benzoic acid, 5-bromo-2-[(2-methylpyridin-4-yl)carbamoyl]benzoic acid, 5-bromo-2-[(2-methylpyrimidin-4-yl)carbamoyl]benzoic acid, 2-[(6-methoxypyridin-2-yl)carbamoyl]-5-(trifluoromethyl)benzoic acid, 2-[(5,6-dimethylpyridin-2-yl)carbamoyl]-5-(trifluoromethyl)benzoic acid, 2-[(5,6,7,8-tetrahydroquinolin-2-yl)carbamoyl]-5-(trifluoromethyl)benzoic acid, or a pharmaceutically acceptable salt, solvate, hydrate, tautomer, optical isomer, N-oxide, and/or prodrug thereof, as disclosed in WO2021/186054 A1.

In another embodiment, the small molecule sortilin inhibitor may also be any of the compounds disclosed in WO2023/101595A1, which is hereby incorporated in its entirety by reference. In particular, the small molecule sortilin inhibitor may be any of the specific compounds recited in claim 12 of WO2023/101595A1, i.e, any of:
(E)-2-[2-(dimethylamino)-4-methyl-5-pyrimidinylcarbonylamino]-5,5-dimethyl-3-hexenoic acid; (E)-5,5-dimethyl-2-1[p-(2,2,2-trifluoroacetyl)benzoylamino]-3-hexenoic acid; (E)-5,5-di-methyl-2-[4-(2-pyridyl)-2-piperazinylcarbonylamino]-3-hexenoic acid; (E)-5,5-dimethyl-2-[4-(4-pyridyl)-2-piperazinylcarbonylamino]-3-hexenoic acid; (E)-5,5-dimethyl-2-(6-phenoxynico-tinoylamino)-3-hexenoic acid; (E)-5,5-dimethyl-2-[m-(4H-1,2,4-triazol-4-yl)benzoylamino]-3-hexenoic acid; (E)-5,5-dimethyl-2-(6-methyl-1H-1,5-diazainden-2-ylcarbonylamino)-3-hexenoic acid; (E)-2-(4-chloro-2-pyrrolylcarbonylamino)-5,5-dimethyl-3-hexenoic acid; (E)-2-(3-iso-butyl-1-methyl-5-pyrazolylcarbonylamino)-5,5-dimethyl-3-hexenoic acid; (E)-5,5-dimethyl-2-[4-(2-pyridyl)-2-pyrrolylcarbonylamino]-3-hexenoic acid; (E)-5,5-dimethyl-2-[4-(4-pyridyl)-2-pyrrolylcarbonylamino]-3-hexenoic acid; (E)-5,5-dimethyl-2-[4-(3-pyridyl)-2-pyrrolylcarbonylamino]-3-hexenoic acid; (E)-2-{4-[amino(2-pyridyl)methyl]-2-pyrrolylcarbonylamino}-5,5-di-methyl-3-hexenoic acid; (E)-2-(3-isobutyl-5-isoxazolylcarbonylamino)-5,5-dimethyl-3-hexenoic acid; (E)-5,5-dimethyl-2-[1-(4-pyridyl)-4-pyrazolylcarbonylamino]-3-hexenoic acid; (E)-5,5-dimethyl-2-[1-(2-oxo-4-pyridyl)-4-pyrazolylcarbonylamino]-3-hexenoic acid; (E)-2-[2-(di-methylamino)-5-pyrimidinylcarbonylamino]-5,5-dimethyl-3-hexenoic acid; (E)-2-(2-isopropyl-5-pyrimidinylcarbonylamino)-5,5-dimethyl-3-hexenoic acid; (E)-2-[2-(dimethylamino)-4-pyrimidinylcarbonylamino]-5,5-dimethyl-3-hexenoic acid; (E)-2-[2-(dimethylamino)-6-methyl-4-pyridinecarbonylamino]-5,5-dimethyl-3-hexenoic acid; (E)-2-[2-(diethylamino)-6-methyl-4-pyrimidinylcarbonylamino]-5,5-dimethyl-3-hexenoic acid; (E)-5,5-dimethyl-2-[p-(1H-1,2,4-triazol-1-yl)benzoylamino]-3-hexenoic acid; (E)-5,5-dimethyl-2-[p-(4H-1,2,4-triazol-4-yl)benzoylamino]-3-hexenoic acid; (E)-5,5-dimethyl-2-[m-(1H-1,2,4-triazol-1-yl)benzoylamino]-3-hexenoic acid; (E)-5,5-dimethyl-2[m-(1-imidazolyl)benzoylamino]-3-hexenoic acid; (E)-5,5-dimethyl-2-[m-(1H-tetraazol-1-yl)benzoylamino]-3-hexenoic acid; (E)-5,5-dimethyl-2-[2-(4H-1,2,4-triazol-4-yl)isonicotinoylamino]-3-hexenoic acid; (E)-2-(4-bromo-2-pyrrolylcarbonyl-amino)-5,5-dimethyl-3-hexenoic acid; (E)-2-(5-chloro-2-pyrrolylcarbonylamino)-5,5-dimethyl-3-hexenoic acid; (E)-2-(4,5-dichloro-2-pyrrolylcarbonylamino)-5,5-dimethyl-3-hexenoic acid; (E)-2-(4-chloro-1-methyl-2-pyrrolylcarbonylamino)-5,5-dimethyl-3-hexenoic acid; (E)-2-(4-chloro-2-thienylcarbonylamino)-5,5-dimethyl-3-hexenoic acid; (E)-2-(5-isopropyl-3-pyrazol-ylcarbonylamino)-5,5-dimethyl-3-hexenoic acid; (E)-5,5-dimethyl-2-[p-(3-pyridyloxy)benzoylamino]-3-hexenoic acid; (E)-5,5-dimethyl-2-(5-phenoxynicotinoylamino)-3-hexenoic acid; (E)-5,5-dirnethyl-2-(2-phenoxyisonicotinoylamino)-3-hexenoic acid; (E)-5,5-di-methyl-2-[p-(2-pyridyloxy)benzoylamino]-3-hexenoic acid; (E)-5,5-dimethyl-2-[m-(2-pyridyloxy)benzoylamino]-3-hexenoic acid; (E)-2-[6-(benzyloxy)nicotinoylamino]-5,5-dimethyl-3-hexenoic acid; (E)-5,5-dimethyl-2-[p-(6-methyl-2-pyrazinyloxy)benzoylamino]-3-hexenoic acid; (E)-5,5-dimethyl-2-[p-(2-pyrimidinyloxy)benzoylamino]-3-hexenoic acid; (E)-2-[m-(5-chloro-2-pyridyloxy)benzoylamino]-5,5-dimethyl-3-hexenoic acid; (E)-2-[6-(p-bromophenoxy)nicotinoylamino]-5,5-dimethyl-3-hexenoic acid; (E)-5,5-dimethyl-2-(2-phenoxy-5-pyrimidinylcarbonylaminc)-3-hexenoic acid; (E)-2-{6-[p-(tert-Butyl)phenoxy]nicotinoylamino}-5,5-dimethyl-3-hexenoic acid; (E)-2-[2-(p-cumenyloxy)isonicotinoylamino]-5,5-dimethyl-3-hexenoic acid; (E)-5,5-dimethyl-2-(5-phenoxy-2-pyrazinylcarbonylamino)-3-hexenoic acid; (E)-5,5-dimethyl-2-[p-(5-methyl-2-pyridyloxy)benzoylamino]-3-hexenoic acid; (E)-2-(2-iso-propyl-5-pyrimidinylcarbonylamino)-5,5-dimethyl-3-hexenoic acid; (E)-5,5-dimethyl-2-[6-(4H-1,2,4-triazol-4-yl)-2-pyridylcarbonylamino]-3-hexenoic acid; (E)-5,5-dimethyl-2-[p-(2-thienyl)benzoylamino]-3-hexenoic acid; (E)-5,5-dimethyl-2-[m-(2-thienyl)benzoylamino]-3-hexenoic acid; (E)-2-[p-(p-chlorophenoxy)benzoylamino]-5,5-dimethyl-3-hexenoic acid; (E)-2-[p-(m-chlorophenoxy)benzoylamino]-5,5-dimethyl-3-hexenoic acid; (E)-5,5-dimethyl-2-[p-(phenoxymethyl)benzoylamino]-3-hexenoic acid; (E)-2-(1-ethyl-3-isobutyl-5-pyrazolylcarbonylamino)-5,5-dimethyl-3-hexenoic acid; (E)-2-(3-chloro-1-methyl-5-pyrazolylcarbonyl-amino)-5,5-dimethyl-3-hexenoic acid; (E)-2-(3-isopropyl-1-methyl-5-pyrazolylcarbonyl-amino)-5,5-dimethyl-3-hexenoic acid; (E)-5,5-dimethyl-2-[1-methyl-3-(3-pyridyl)-5-pyrazolylcarbonylamino]-3-hexenoic acid; (E)-2-[3-(3,4-dichlorophenyl)-1-methyl-5-pyrazolylcarbonylamino]-5,5-dimethyl-3-hexenoic acid; (E)-2-[5-(3,4-dichlorophenyl)-1-methyl-3-pyrazolylcarbonylamino]-5,5-dimethyl-3-hexenoic acid; (E)-2-(4,5-dichloro-2-thienylamino)-5,5-di-methyl-3-hexenoic acid; (E)-2-(2-benzyl-4-methyl-5-pyrimidinylcarbonylamino)-5,5-dimethyl-3-hexenoic acid; (E)-5,5-dimethyl-2-[4-methyl-2-(p-toluidino)-5-pyrimidinylcarbonylamino]-3-hexenoic acid; (E)-2-[2-(p-chlorophenylamino)-4-methyl-5-pyrimidinylcarbonylamino]-5,5-di-methyl-3-hexenoic acid; (E)-2-[6-(p-chlorophenoxy)nicotinoylamino]-5,5-dimethyl-3-hexenoic acid; (E)-5,5-dimethyl-2-[16-(p-tolyloxy)nicotinoylamino]-3-hexenoic acid; (E)-2-(6-benzylnicotinoylamino)-5,5-dimethyl-3-hexenoic acid; (E)-2-(p-benzylbenzoylamino)-5,5-dimethyl-3-hexenoic acid; (E)-5,5-dimethyl-2-(p-phenoxybenzoylamino)-3-hexenoic acid; (E)-5,5-dimethyl-2-(6-phenylnicotinoylamino)-3-hexenoic acid; (E)-5,5-dimethyl-2-(2-phenylisonicotinoylamino)-3-hexenoic acid; (E)-5,5-dimethyl-2-[6-(2-thienyloxy)nicotinoylamino]-3-hexenoic acid; (E)-2-(5-chloro-1-benzothiophen-2-ylcarbonylamino)-5,5-dimethyl-3-hexenoic acid; (E)-2-(5-chloro-2-indolylcarbonylamino)-5,5-dimethyl-3-hexenoic acid; (E)-5,5-dimethyl-2-(1-thia-5-aza-2-indenylcarbonylamino)-3-hexenoic acid; (E)-2-(2-chloro-1-benzothiophen-6-ylcarbonylamino)-5,5-dimethyl-3-hexenoic acid; (E)-2-(2-chloro-6-indolylcarbonylamino)-5,5-dimethyl-3-hexenoic acid; (E)-2-(4-chloro-1,3-thiazol-2-ylcarbonylamino)-5,5-dimethyl-3-hexenoic acid; (E)-2-(4,5-dichloro-1,3-thiazol-2-ylcarbonylamino)-5,5-dimethyl-3-hexenoic acid; (E)-2-(3-chloro-5-isothiazolylcarbonylamino)-5,5-dimethyl-3-hexenoic acid; (E)-2-(3,4-dichloro-5-isothiazolylcarbonylamino)-5,5-dimethyl-3-hexenoic acid; (E)-5,5-dimethyl-2-[m-(3-pyridyl)benzoylamino]-3-hexenoic acid; (E)-2-[m-(6-chloro-2-methyl-3-pyridyl)benzoylamino]-5,5-dimethyl-3-hexenoic acid; or an enantiomer thereof, a diastereomer thereof, a racemate thereof, a prodrug thereof, or a pharmaceutically acceptable salt thereof.

In another embodiment, the small molecule sortilin inhibitor may also be any of the compounds disclosed in WO2023/031440A1, which is hereby incorporated in its entirety by reference. In particular, the small molecule sortilin inhibitor may be any of the specific compounds recited in claim 12 of WO2023/031440A1.

In another embodiment, the small molecule sortilin inhibitor may also be any of the compounds disclosed in WO2023/161505A1, which is hereby incorporated in its entirety by reference. In particular, the small molecule sortilin inhibitor may be any of the specific compounds recited in claim 9 of WO2023/161505A1, i.e., any of (2S)-2-[(benzylcarbamoyl)amino]-5,5-dimethylhexanoic acid, (2S)-2-{[benzyl(methyl)carbamoyl]amino}-5,5-dimethylhexanoic acid, (2S)-2-{[(benzyloxy)carbonyl]amino}-5,5-dimethylhexanoic acid, (2S)-5,5-dimethyl-2-[(morpholine-4-carbonyl)amino]hexanoic acid, or (2S)-5,5-dimethyl-2-[(1,4-oxazepane-4-carbonyl)amino]hexanoic acid.

In another embodiment, the small molecule sortilin inhibitor may also be any of the compounds disclosed in WO2024/047227A1, which is hereby incorporated in its entirety by reference. In particular, the small molecule sortilin inhibitor may be any of the specific compounds recited in claim 9 or claim 12 of WO2024/047227A1.

In another embodiment, the small molecule sortilin inhibitor may also be any of the compounds disclosed in EP4089102A1, which is hereby incorporated in its entirety by reference. In particular, the small molecule sortilin inhibitor may be any of the specific compounds recited in claim 10 of EP4089102A1.

In another embodiment, the small molecule sortilin inhibitor may also be any of the compounds disclosed in WO2022/223805A1, which is hereby incorporated in its entirety by reference. In particular, the small molecule sortilin inhibitor may be any of the specific compounds recited in claim 10 of WO2022/223805A1.

In another embodiment, the small molecule sortilin inhibitor may also be any of the compounds disclosed in WO2022/157271A1, which is hereby incorporated in its entirety by reference. In particular, the small molecule sortilin inhibitor may be any of the specific compounds recited in claim 9 of WO2022/157271A1.

Additional small molecule inhibitors of sortilin are disclosed in Stachel et al., 2020, which is hereby incorporated by reference. Thus, the sortilin inhibitor may also have a formula of, e.g.:

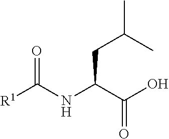

wherein R¹ may be selected from any of:

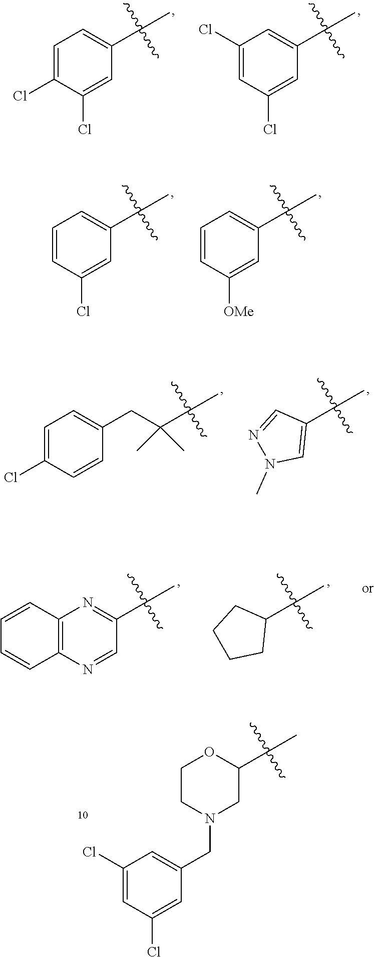

The small molecule inhibitor may also have a formula of:

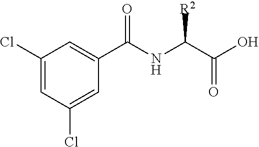

wherein R² may be selected from any of:

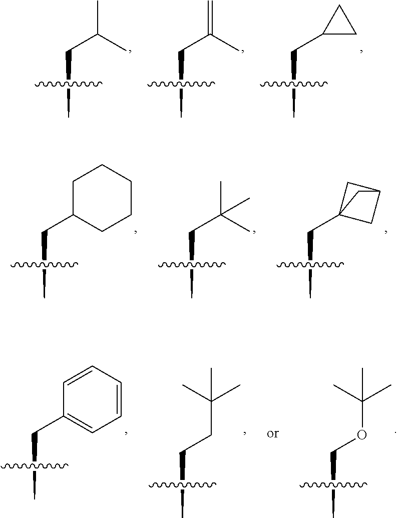

Alternatively, the small molecule inhibitor of sortilin may have a formula of:

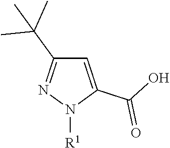

wherein R¹ may be selected from any of:

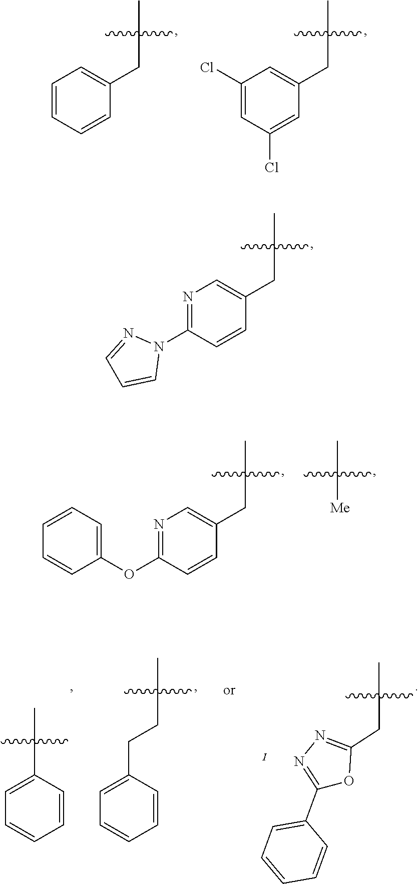

The small molecule inhibitor of sortilin may also have a formula of:

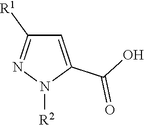

wherein R¹ is

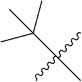 [SMILES: *C(C)(C)C]

and R² is phenethyl; or
wherein R¹ is

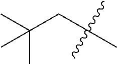 [SMILES: *CC(C)(C)C]

and R² is benzyl; or
wherein R¹ is

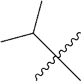 [SMILES: *C(C)C]

and R² is phenethyl; or
wherein R¹ is

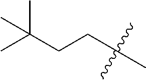 [SMILES: *CCC(C)(C)C]

and R² is benzyl.

The small molecule inhibitor may also be a sortilin inhibitor as disclosed in Anand et al., 2023, which is herewith incorporated by reference as well.

The small molecule inhibitor may also be obtainable by a method for identifying a sortilin ligand as disclosed in WO2009132656A2, which is herewith incorporated by reference.

The sortilin inhibitor may be any of the herein described small molecules. However, in some embodiments, the sortilin inhibitor may comprise not only a single type of small molecule but a combination of several different small molecule drugs targeted against sortilin, e.g., it may comprise a combination of any of the herein described small molecule inhibitors such as, e.g., a combination of AF38469 and MPEP, of AF38469 and AF40431 or of AF38469, AF40431 and MPEP.

In alternative embodiments, the sortilin inhibitor may be an anti-sortilin antibody. An antibody (also known as immunoglobulin) is a natural or synthetic, typically Y-shaped protein that can selectively bind a target antigen. The term “antibody” may include both polyclonal and monoclonal antibodies, however, preferably, the anti-sortilin inhibitor is a monoclonal antibody. In addition to complete immunoglobulin molecules comprising both variable and constant regions, an antibody in the context of the invention may also be a fragment of an antibody, e.g., a Fab fragment or a Fab₂ fragment, a human or humanized antibody, a bivalent antibody or a single chain antibody, as long as it selectively binds the target antigen. The antibody may be chosen or designed using standard laboratory techniques to specifically bind to an epitope of sortilin in a fashion that blocks binding of a sortilin ligand to sortilin.

Accordingly, in cases where the sortilin inhibitor is an anti-sortilin inhibitor, it preferably functions as an antagonistic antibody.

At present, at least two suitable anti-sortilin antibodies are well known from the art and are currently tested in clinical trials.

The first of these antibodies, AL001, is also known under the name Latozinemab. It is disclosed, amongst other anti-sortilin antibodies, in WO2020014617A1, which is hereby incorporated by reference. AL001 has been successfully tested for the treatment of frontotemporal dementia and/or ALS in Phase 2 and Phase 3 clinical trials, including NCT04374136, NCT03987295, NCT03636204 and NCT05053035. AL001 comprises a heavy chain having an amino acid sequence of SEQ ID NO: 1 and a light chain having an amino acid sequence of SEQ ID NO: 2.

Thus, in a preferred embodiment, the sortilin inhibitor of the present invention is an anti-sortilin antibody having a heavy chain with an amino acid sequence of SEQ ID NO: 1 and a light chain with an amino acid sequence of SEQ ID NO: 2.

The second anti-sortilin antibody that tested in a Phase 1 clinical trial (NCT04111666) is known under the name AL101 and is disclosed in WO2022120352A1, which is herewith incorporated by reference. AL101 comprises a light chain variable domain and a heavy chain variable domain, wherein the heavy chain variable domain comprises an HVR—H1 comprising the amino acid sequence of SEQ ID NO:3, an HVR-H2 comprising the amino acid sequence of SEQ ID NO: 4, and an HVR-H3 comprising the amino acid sequence of SEQ ID NO:5, and the light chain variable domain comprises an HVR-L1 comprising the amino acid sequence of SEQ ID NO:6, an HVR-L2 comprising the amino acid sequence of SEQ ID NO: 7, and an HVR-L3 comprising the amino acid sequence of SEQ ID NO:8. The anti-sortilin antibody may thus also comprise a light chain comprising the amino acid sequence of SEQ ID NO: 15, and a heavy chain comprising the amino acid sequence of SEQ ID NO: 16 or 17. It can also be used in the context of the invention.

Other anti-sortilin antibodies have been described in the prior art. For instance, the anti-sortilin inhibitor may also be any of the other anti-sortilin antibodies besides AL001 that are disclosed in WO2020014617A1. The anti-sortilin antibody may also be any of the anti-sortilin antibodies disclosed in VVO2016/164637A1, which is hereby incorporated in its entirety by reference. Alternatively, the sortilin inhibitor may also be an anti-sortilin antibody as disclosed, e.g., in Miyakawa et al., 2020, which is hereby incorporated by reference as well.

In some embodiments, the sortilin inhibitor may comprise a combination of two or more different anti-sortilin antibodies, i.e, a combination of anti-sortilin antibodies that bind to different epitopes of the sortilin protein. For instance, the sortilin inhibitor may comprise a mixture of the anti-sortilin antibodies AL001 and AL101.

In further embodiments, the sortilin inhibitor may be an inhibitory nucleic acid inhibiting the expression of sortilin, wherein, typically, the inhibitory nucleic acid is an inhibitory RNA.

Most inhibitory RNAs rely on a process known as RNA interference (RNAi) to post-transcriptionally inhibit the expression of a gene of interest. During this process, the inhibitory RNA guides a protein complex known as RNA-induced silencing complex (RISC) to a target mRNA molecule that has been transcribed from the gene of interest. Binding of said target mRNA is mediated via complementary base pairing between the inhibitory RNA and the target mRNA and is thus sequence-specific. Upon binding to the target mRNA, the protein complex associated with the inhibitory RNA initiates mRNA degradation or inhibits its translation into protein.

The inhibitory RNA can be, e.g., a short hairpin RNA (shRNA), a microRNA (miRNA) or a small interfering RNA (siRNA). MicroRNAs (miRNAs) are single-stranded, non-coding RNAs that are generated from longer endogenous precursor transcripts forming hairpin structures and are processed through endonucleolytic cleavage into short RNA duplexes of 20-23 base pairs (bp). Typically, only the antisense strand of these duplexes is incorporated into RISC to guide the complex to a complementary target RNA. Naturally occurring small interfering RNAs (siRNAs) rely on a similar biogenesis pathway as miRNAs but originate from double-stranded RNA molecules rather than from hairpin structures. For instance, in many cases, siRNAs are naturally produced from double-stranded viral RNA. Mature siRNAs typically have a length of 20 to 25 nt and, similar to miRNAs, rely on RNAi to prevent target RNAs from being translated into protein. However, whereas naturally occurring miRNAs typically silence genes by repression of translation and have broader specificity of action, naturally occurring siRNAs typically exhibit a higher specificity as they oftentimes bind with up to 100% sequence complementarity to their target sequences. Many siRNAs thus can induce cleavage of their target mRNA via RISC before translation. Shortly after the discovery of the RNAi pathway, scientist have recognized the therapeutic potential of carefully designed synthetic siRNA-based drugs. The first siRNA therapeutic, patisiran, was approved by the FDA in 2018 and many more are expected to follow. Site-specific delivery typically requires the use of suitable delivery systems and/or chemical modifications that can stabilize the siRNA itself, e.g., as known in the art.

Short hairpin RNAs (shRNA) constitute an alternative to siRNA-based therapeutics. shRNAs are artificially designed single stranded RNA molecules that exhibit a characteristic hairpin turn. shRNAs typically have a length of 50-70 nt and form a stem-loop structure consisting of a 19 to 29 bp region of double-strand RNA (the stem) bridged by a region of predominantly single-stranded RNA (the loop) and a dinucleotide 3′ overhang. Once they are introduced into a cell, they are processed similar to miRNAs into short RNA duplexes that resemble siRNAs. One strand of this duplex is then incorporated into RISC to facilitate knock-down of a target gene. ShRNAs can be encoded by an expression vector, such es a viral vector, which allows expression and processing of the shRNA construct inside a target cell.

ShRNAs are normally transcribed by RNA polymerase Ill. However, the expression of exogenous shRNAs in cells can saturate endogenous miRNA signaling pathways and has been associated with toxic effects. Moreover, imprecise processing of the stem loop structures of shRNAs may result in aberrant guide- and passenger-strand mediated off-target effects. To overcome these problems, shRNAs are preferably provided as a “shRNAmiRs”, i.e., the shRNA is embedded into a framework or backbone of a miRNA. Such an shRNAmiR may also be referred to as an artificial miRNA or miRNA mimic. shRNAmiRs can be generated from Polymerase II promoters, enabling constitutive, tissue specific or inducible expression (Adams et al., 2017).

A person skilled in the art is aware of how to choose or specifically design an inhibitory RNA capable of binding and repressing an mRNA that encodes sortilin by using standard laboratory techniques. For instance, siRNA-mediated knockdown of sortilin has been repeatedly achieved in the literature, e.g. in Lefrancois et al., 2005; Roselli et al, 2015, Uchiyama et al., 2017 and Gao et al., 2020. Accordingly, in case the sortilin inhibitor is an siRNA, the siRNA may have a nucleic acid sequence as disclosed in any of these publication, i.e. the siRNA targeted against sortilin may comprise or consist of a nucleic acid sequence of any of, e.g., SEQ ID NOs: 9-14 (9: AGGTGGTGTTAACAGCAGAG, 10: AATGTTCCAATGCCCCACTC, 11: CUCUGCUGUUAACACCACC[dT][dT], 12: UUUACAACCUAUGCAAAUGG, 13: CCAAGUCAAAUUCUGUCCCUAUUAU, 14: GAGAACUCUGGAAAGGUGAUACUAA).

In case the sortilin inhibitor is an inhibitory RNA, the RNA may comprise at least one modification to increase its stability. The skilled person is aware of various such stabilizing modifications that may be introduced into, e.g., an siRNA. Suitable modifications are summarized e.g., in Selvam et al., 2017.

In an alternative embodiment, the skilled person may choose to design an inhibitory nucleic acid that is not necessarily an RNA, e.g., it may be an antisense oligonucleotide targeted against the mRNA of sortilin to prevent its expression. Antisense oligonucleotides are short synthetic nucleic acids made either from DNA or RNA that sequence-specifically bind to target mRNAs via complementary base pairing to block their translation into protein. As in the case of inhibitory RNAs for use in RNAi-mediated gene knockdown, a person skilled in the art is aware of standard laboratory techniques well known to the field to choose or design appropriate antisense oligonucleotides that specifically target the mRNA of sortilin.

The inhibitory nucleic acid may also be an aptamer against sortilin, i.e., an artificial oligonucleotide made of DNA, RNA or XNA that, due to its specific 3-dimensional structure, can bind with high affinity to the sortilin protein to cause its inhibition. XNA (short for xenobiotic nucleic acid) refers to a synthetic nucleic acid analogue. The skilled person will be aware of types of XNAs suitable for aptamer production. For instance, the XNA-aptamer may consist of 5-anhydrohexitol nucleic acid (HNA). In some embodiments, the aptamer may be an L-ribonucleic acid aptamer (also known by its tradename Spiegelmer). Spiegelmers are artificial RNA oligonucleotides built of L-ribose rather than D-ribose, thus rendering them highly resistant to degradation by nucleases. Like conventional aptamers, Spiegelmers directly bind to their target proteins to mediate their functional inhibition.

In one embodiment, the sortilin inhibitor is a single type of inhibitory nucleic acid, e.g., it may be an siRNA having or comprising any of the sequences disclosed herein. However, in other embodiments, the sortilin inhibitor may be a composition comprising a mixture of different inhibitory nucleic acids. For instance, the sortilin inhibitor for the herein described use may comprise an inhibitory RNA, e.g., one or more the siRNA molecules described herein as well as an antisense oligonucleotide directed to sortilin mRNA. The sortilin inhibitor may also comprise a mixture of different siRNA constructs directed to sortilin mRNA, e.g., the sortilin inhibitor may comprise a mixture of siRNAs of any of the herein described SEQ ID NO.: 9-14.

In yet another embodiment, the sortilin inhibitor may be a peptide-based inhibitor, i.e., it may be a therapeutic peptide that is composed of a series of amino acids. It may have a molecular weight that is bigger than that of a small molecule as defined herein. Accordingly, a peptide-based inhibitor within the meaning of the present invention typically has a molecular weight of more than 800 Dalton, e.g., of 800 to 10,000 Dalton or 2000 to 5000 Dalton. The term “peptide-based inhibitor” may also refer to a soluble fragment of sortilin, or a sortilin-related molecule, which can bind competitively to biologically available sortilin ligands such as pro-neurotrophins. As a result, the sortilin inhibitor results in an inhibition of receptor activation, which would otherwise occur as a result of ligand binding.

In a further embodiment, the sortilin inhibitor may also be proteolysis-targeting chimera (PROTAC). PROTACs consist of two different protein-binding molecules that are covalently connected to each other via short linker region. One of the two protein-binding molecules is capable of binding to a target protein meant for degradation, i.e., in the present case to sortilin. The second molecule binds to an E3 ubiquitin ligase. Accordingly, PROTACs are able to bring the target protein and the E3 ligase into close proximity so that the target protein can be ubiquitinated and thus marked for proteasomal degradation. The skilled person will be able to develop effective PROTACs against sortilin based on the known prior art. This is because, in contrast to small molecule inhibitors, PROTACs need only bind to a target protein with high selectivity, without, however, having to exert an inhibitory effect on the enzymatic activity of said target protein. For targeting, an antibody to sortilin or a derivative thereof, e.g., a scFv may be used.

In some embodiments, the sortilin inhibitor may also be a combination of any of the different types of inhibitors described herein. For instance, the sortilin inhibitor may comprise a mixture of an anti-sortilin antibody such as AL001 or AL101 and/or a small molecule inhibitor such as AF38469.

In a particularly preferred embodiment, the condition associated with the release of an endocrine and/or a paracrine factor by a functional neuroendocrine tumor is carcinoid syndrome, the endocrine and/or a paracrine factor is serotonin and the sortilin inhibitor is 2-[[(6-methyl-2-pyridinyl)amino]carbonyl]-5-(trifluoromethyl)-benzoic acid or an anti-sortilin antibody having a heavy chain with an amino acid sequence of SEQ ID NO: 1 and a light chain with an amino acid sequence of SEQ ID NO: 2. Preferably, it is 2-[[(6-methyl-2-pyridinyl)amino]carbonyl]-5-(trifluoromethyl)-benzoic acid.

The sortilin inhibitor may be provided in a suitable pharmaceutical composition, wherein said pharmaceutical composition may comprise additional ingredients such as, e.g., a pharmaceutically acceptable excipient. The term “pharmaceutically acceptable” refers those compounds, materials, compositions, and/or dosage forms that are, within the scope of sound medical judgement, suitable for use in contact with the tissues of the subject without causing excessive toxicity, irritation, allergic response or other problems or complications commensurate with a reasonable benefit/risk ratio.

In many embodiments, the pharmaceutical composition further comprises a suitable carrier, i.e., a compound, composition, structure or substance that, when in combined with the sortilin inhibitor described herein, aids or facilitates preparation, storage, administration, delivery, effectiveness, selectivity, or any other feature of the inhibitor for its intended use or purpose. It may e.g. comprise water, a saline, e.g., physiological saline, or a buffer.

In one embodiment, said carrier is a vector comprising or encoding the sortilin inhibitor. The type of vector depends primarily on the nature of the inhibitor used to inhibit sortilin. For instance, in particular if the sortilin inhibitor is a small molecule inhibitor, an anti-sortilin antibody, an in-vitro generated inhibitory nucleic acid, a PROTAC or a peptide-based inhibitor, the vector used for transporting the sortilin inhibitor to its site of action may be a lipid vesicle or comprise a lipid membrane that encapsulates the inhibitor. For example, the vector may be a liposomal or an exosomal carrier comprising the sortilin inhibitor as a cargo and, optionally, expressing a targeting moiety on its surface. The vector may also be a lipid nanoparticle. Alternatively, the vector carrying the sortilin inhibitor may be a bacterial minicell. These types of carriers have the ability to entrap both hydrophilic therapeutic agents within their central aqueous core or lipophilic drugs within their bilayer compartment. The targeting moiety may be, e.g., a cell-specific antibody or ligand such as, e.g., a small peptide, polysaccharide or nucleic acid that can be bound by a receptor expressed on the surface of the cell of interest.

In another embodiment, the vector may be a polymeric micro- or nanoparticle. The micro- or nanoparticle may be formed of any physiologically acceptable polymeric material known in the art, e.g., it may comprise a biopolymer such as polysaccharide selected from the group comprising cellulose, alginate or chitosan. The vector may also be a nanocarrier comprising, e.g., a hyperbranched dendritic polyglycerol polymer. Such a nanocarrier is an amphiphilic unimolecular nanocarrier of dendritic structure, and it comprises a dendritic core and at least one shell.

In case the sortilin inhibitor is an inhibitory nucleic acid that is not in vitro-transcribed, it is inserted into an expression vector encoding the inhibitory nucleic acid. Examples of such expression vectors include but are not limited to minicircles, plasmids, cosmids, phages, viruses or artificial chromosomes. In some embodiments, the vector preferably is a viral vector, e.g., a lentiviral vector, an adenoviral vector or an adeno-associated viral vector. Expression vectors typically contain a cargo sequence comprising an expression cassette, i.e. the necessary elements that permit transcription of a nucleic acid into inhibitory nucleic acid, such as a suitable promoter. A person skilled in the art will be able to design suitable expression vectors to comprise a cargo DNA sequence encoding the inhibitory nucleic acid based on general knowledge and the prior art. For example, a stretch of cargo DNA, i.e., a transgene, encoding an inhibitory RNA may be operably linked to a suitable promoter and inserted into the expression vector of choice. The promoter used to express the inhibitory RNA may be any constitutively active promoter known in the art, e.g., a Rous sarcoma virus (RSV) promoter, a human cytomegalovirus (CMV) promoter, an HIV promoter or a eukaryotic promoter such as e.g., EF1a, PGK1, UBC, or human beta actin. Alternatively, the promoter may be an inducible promoter that only drives expression of the inhibitory RNA in the presence or absence of a certain stimulus. For example, the inducible promoter may be a Tet-regulated promoter, i.e., it may comprise a tetracycline response element (TRE) that can be bound by a tetracycline transactivator (tTA) protein in the presence of tetracycline or an analogue thereof, e.g. doxycycline. The promoter may also be a cell-specific promoter which only drives expression in a particular type of cell, in the present case, in the NET of interest.

In case a viral vector is used for expression of the inhibitory RNA, the assembled viral vector is preferably pseudotyped, e.g., with a viral envelope glycoprotein or a capsid capable of binding to a receptor expressed on the surface of the cell of interest. The term pseudotyping refers to the generation of viral vectors that carry foreign viral envelope glycoproteins on their surface or are encapsulated in a capsid of a different virus or viral serotype to specifically target only particular cell types of interest. The viral vector particles are usually assembled inside suitable packaging cells known in the art according to standard laboratory techniques before they are administered to the subject.

Inhibitory nucleic acids may also be delivered to the cells of interest using a vector that is a non-pathogenic, genetically modified bacterium or yeast comprising a prokaryotic or eukaryotic vector. The vector comprises a DNA molecule encoding the inhibitory nucleic operably linked to a promoter that controls expression of said inhibitory nucleic acid, as described above. The non-pathogenic bacterium is preferably invasive, i.e., it is capable of entering a host cell and may be derived, e.g., from a non-pathogenic strain of Escherichia coli, Listeria, Yersinia, Rickettsia, Shigella, Salmonella, Legionella, Chlamydia, Brucella, Neisseria, Burkholderia, Bordetella, Borrelia, Coxiella, Mycobacterium, Helicobacter, Staphylococcus, Streptococcus, Vibrio, Lactobacillus, Porphyromonas, Treponema, or Bifidobacteriae. Preferably, the prokaryotic or eukaryotic vector is specifically designed for targeted delivery to a cell of interest, e.g., by expressing a ligand on its surface that can be recognized by a receptor on the target cell.

Alternatively, the inhibitory nucleic is not expressed on site from an expression vector at the cell of interest, but instead is expressed in vitro before being packaged into a suitable vector vehicle, e.g., as described above, and directly delivered to the target cell. This way, the time of nucleic acid-mediated target gene inhibition can be effectively limited.

The sortilin inhibitor according to the invention may be administered directly to the subject in need thereof, e.g., orally, intravenously, intramuscularly, intraperitoneally, subcutaneously, transdermally or transmucosally. Administration may be by a single administration, e.g., an injection or infusion. A slow-release formulation may also be chosen if administration over a longer time period is desired. Oral administration may be in a form suitable for targeted release in a specific area of the gut, e.g., depending on the location of the NET.

According to the method of the invention, a therapeutically effective amount of the sortilin inhibitor is to be administered to the subject. In the context of the invention, “a therapeutically effective” amount refers to the amount of the inhibitor that is of sufficient quantity to ameliorate one or more causes or symptoms of a condition as described herein. Such amelioration only requires a reduction or alteration, not necessarily elimination.

A person skilled in the art will be able to choose a suitable vector depending on the type of inhibitor used, the route of administration, the respective target cell to which the inhibitor is to be delivered and the condition to be treated. In addition, based on the known state of the art, the skilled person will be able to further modify the vector for optimized targeted delivery and release of the inhibitor.

Throughout the invention, the term “about” is intended to be understood as “+/−10%”. If “about” relates to a range, it refers to both lower and upper limits of the range. “A” is intended to mean “one or more”, if not explicitly mentioned otherwise.

All literature cited herein is herewith fully incorporated. The present invention is further illustrated, but not limited, by the following examples.

### FIGURE LEGENDS

FIG. 1: Sortilin is a marker of functional neuroendocrine tumors (NETs). (A) Representative images of a pancreatic NET stained for sortilin (top panels) or by using secondary antibody only (control, bottom panels) (B) Representative images of a small intestinal NET stained for sortilin (top panels) or by using secondary antibody only (control, bottom panels) (C)-(F) Staining intensity was estimated as described in Kim et al. 2018 by an immunoreactivity score, which was defined by the product of the value for immunostaining intensity (0, no staining; 1, weak staining; 2, moderate staining; 3, strong staining) and for the percentage of positive immunoreactive cells (0, no positive; 1, 0%-10% positive; 2, 11%-50% positive; 3, 51%-100% positive). Every sample consisted of three replicates. By averaging each immune reactivity score, the final score for each sample was determined. (G) Western Blot for sortilin in three non-function (nf) and three functional (f) liver metastasis of well differentiated small intestinal NETs.

FIG. 2: Sortilin is expressed in neuroendocrine tumor cell cultures. Western blot for assessing sortilin expression in non-neuroendocrine (Hela) cell culture as well as two neuroendocrine tumor cell cultures (QGP1 and BON).

FIG. 3: Concentration of amines in BON cell lysates and supernatants after treatment with sortilin inhibitor

(A)-(D) BON neuroendocrine tumor cells were incubated for 23 h in serotonin free medium with (Sort Inh) or without (Control) the sortilin inhibitor AF38469 (10 μM). Afterwards, the serotonin (5HT, A), Tryptophan (TRP, B) and Tyrosin (TYR, C) content as well as the content of the serotonin metabolite 5-hydroxyindoleacetic acid (5-HIAA) in the cell culture supernatant was assessed using high performance liquid chromatography (HPLC). The cells were treated with fresh sortilin inhibitor for two more hours. Subsequently, the 5HT, (E, F), TRP, (G, H) and TYR (I, J) content in the cell lysates was measured in the presence or absence of the secretion inductor of monoamines Paramethoxyamphetamin (PMA) via HPLC as well.

FIG. 4: Proliferation of BON cells after sortilin inhibition. To exclude the possibility of toxic effects of sortilin inhibition on BON cells, a cell proliferation assay was performed using the Alamar Blue cell viability assay 5 days after sortilin inhibition.

FIG. 5: Sortilin is expressed in healthy human tissues. (A) Western blot analysis demonstrates expression of sortilin protein in healthy human colon and ileum tissues as well as in a functional neuroendocrine tumor. (B) Representative image of healthy human pancreas tissue immunostained for sortilin. (C) Representative image of healthy human colon tissue immunostained for sortilin. Immunostaining revealed that the endocrine part of the pancreas (the pancreatic islet) was highly positive for sortilin. Staining the midgut for sortilin revealed scattered sortilin positive cells resembling neuroendocrine cells.

FIG. 6: Sortilin is expressed in neuroendocrine cells. Double immunostaining for sortilin and synaptophysin (marker of neuroendocrine cells) in murine ileum (A) and colon (B) confirmed that sortilin is expressed in neuroendocrine cells.

FIG. 7: Effect of sortilin inhibition on serotonin content of neuroendocrine organoids. (A) Experimental scheme for testing the effects of sortilin inhibition on serotonin levels in neuroendocrine organoids. Murine intestinal organoids are generated and enriched with neuroendocrine cells as previously described in (Kawasaki et al, 2018; Basak et al, 2017). Enrichment with neuroendocrine cell is demonstrated by immunostaining for synaptophysin. Organoids are subsequently incubated four days in the absence (control) or the presence of the sortilin inhibitor AF38469 (10 μM). The lysates of these organoids are next harvested, and serotonin levels are measured by HPLC. (B) Organoids treated with the sortilin inhibitor (Sort Inh) showed a lower concentration of serotonin (5HT) per cell. This result underlines the role of sortilin in hormone production in neuroendocrine cells and underpins sortilin as a promising therapeutic target.

FIG. 8: Impact of sortilin deficiency on splenic amine concentration in sortilin knockout mice. A sortilin knockout (KO) mouse model was generated previously (Jansen et al., 2007) by deleting 41 codons in exon 14 of the Sort1 gene. Sortilin KO mice exhibited reduced serotonin (5HT, A), Tryptophan (Trp, B) and Tyrosine (Tyr, C) levels in their spleens compared to wild type (WT) mice.

### EXAMPLES

Sortilin, also known as neurotensin receptor 3, is a member of the VPS10P domain sorting receptor family, a group of transmembrane receptors which are involved in sorting and trafficking of a broad range of ligands (Malik et al., 2020). It has been already shown that sortilin is expressed in many healthy as well as in many cancer cells like breast cancer or lung cancer (Roselli et al., 2015; Al Akhrass et al., 2017; Gao et al., 2018). Expression was also found in NETs and was attributed to cell migration and adhesion (Kim et al., 2018). However, only a fraction of the stained tissue samples was positive for sortilin expression (Kim et al., 2018).

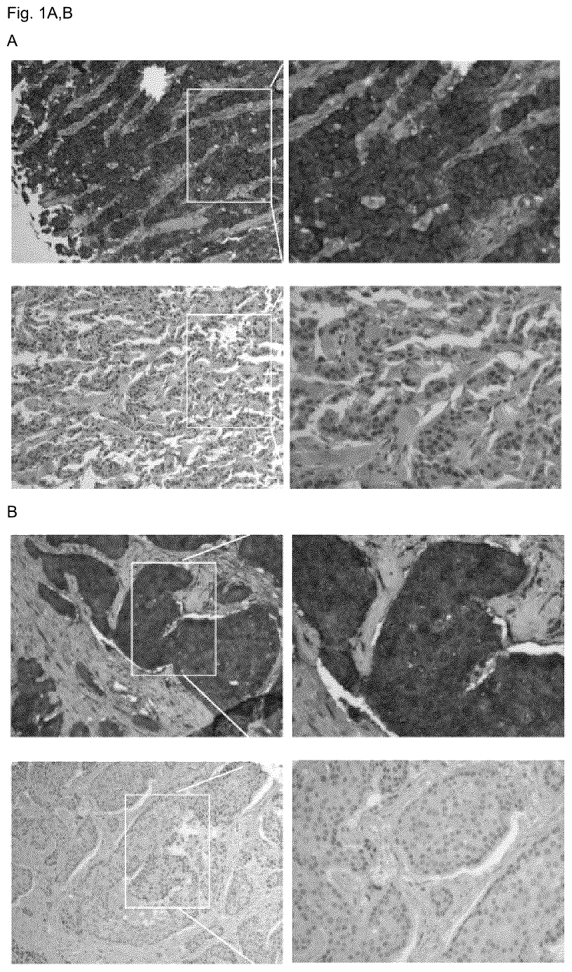

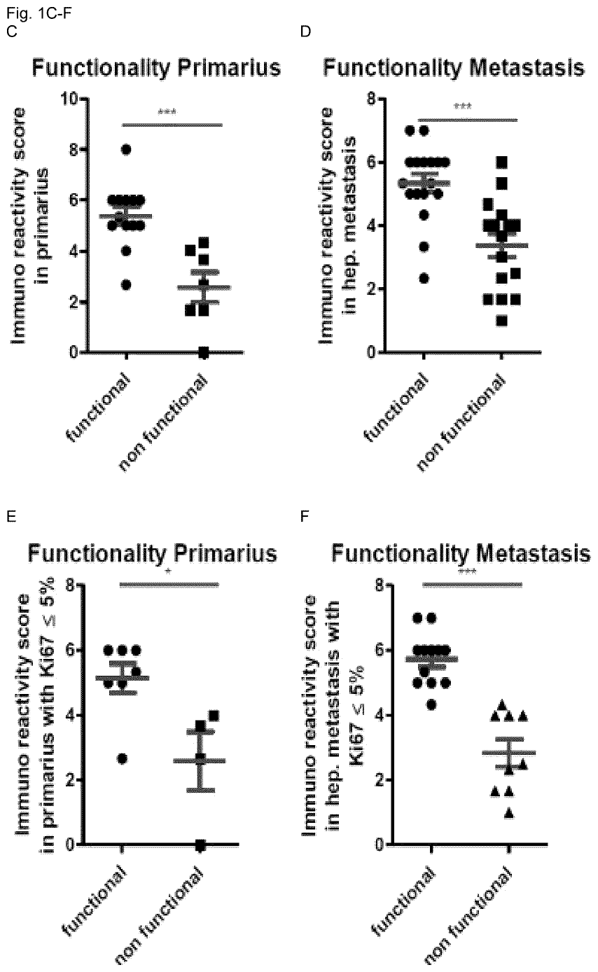

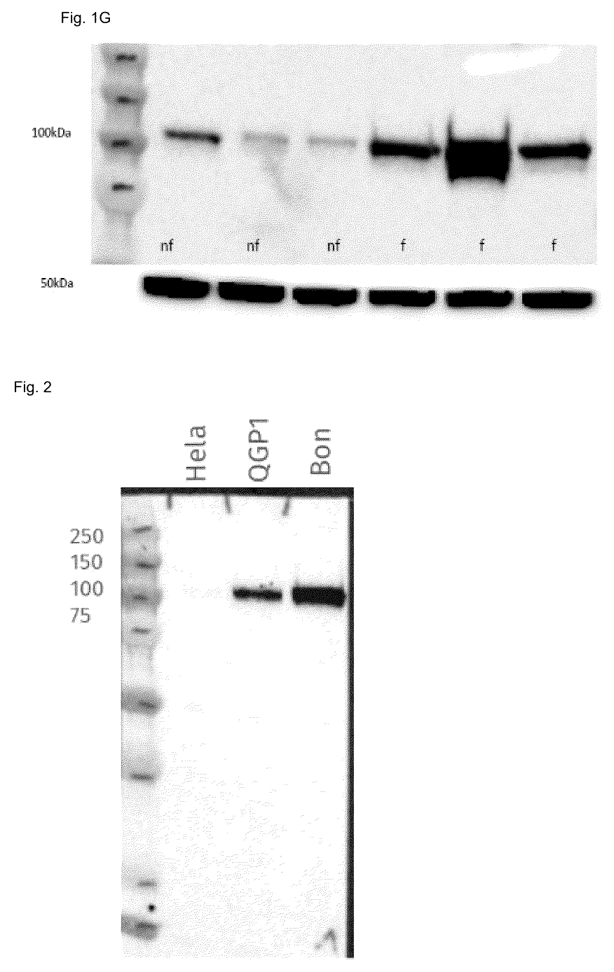

We stained by immunohistochemical means 52 NET samples for sortilin (used antibody: R&D systems: AF3154) (FIG. 1A, B). In a total of 21 intestinal or pancreatic primary tumors and 31 hepatic metastases of intestinal, pancreatic or lung (1 sample) NETs were analyzed. All immunostained primary tumors also had hepatic metastases. Staining intensity was estimated by an immunoreactivity score, which was defined by the product of the value for immunostaining intensity (0, no staining; 1, weak staining; 2, moderate staining; 3, strong staining) and for the percentage of positive immunoreactive cells (0, no positive; 1, 0%-10% positive; 2, 11%-50% positive; 3, 51%-100% positive). This score was taken similar to previous publications (Kim et al., 2018). Every sample consisted of three replicates. By averaging each immune reactivity score we obtained the final score for each sample. For each replicate a negative control with a secondary antibody alone was made (FIG. 1C-F).

We could confirm the observation of a high immunoreactivity in a subpopulation of NETs. Interestingly, functional tumors exhibited a significant higher immunoreactivity indicating a higher expression of sortilin in tumors causing a hormone associated, functional tumor stage (FIG. 1C-F). This result persisted also after correction for the proliferation index of the NET samples.

To validate this finding, we performed Western blot analyses of six distinct liver metastases of small intestinal NETs (antibody used: BD Biosciences: #612101). Again, we could confirm that functional NETs express a higher level of sortilin (FIG. 1G).

Thus, we could clearly demonstrate by immunohistochemistry and also Western blotting that sortilin is a marker of functional NETs. To our knowledge this finding represents the first marker which distinguishes functional from nonfunctional tumors.

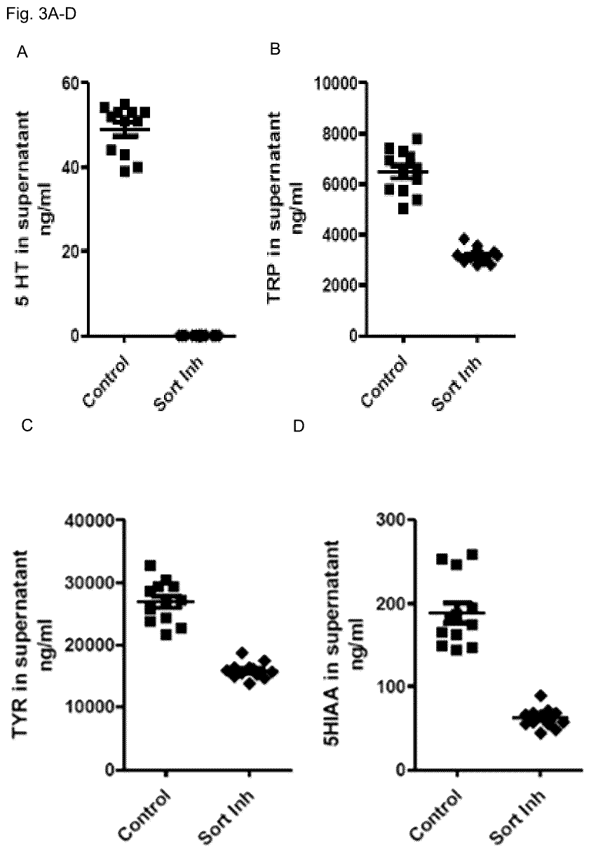

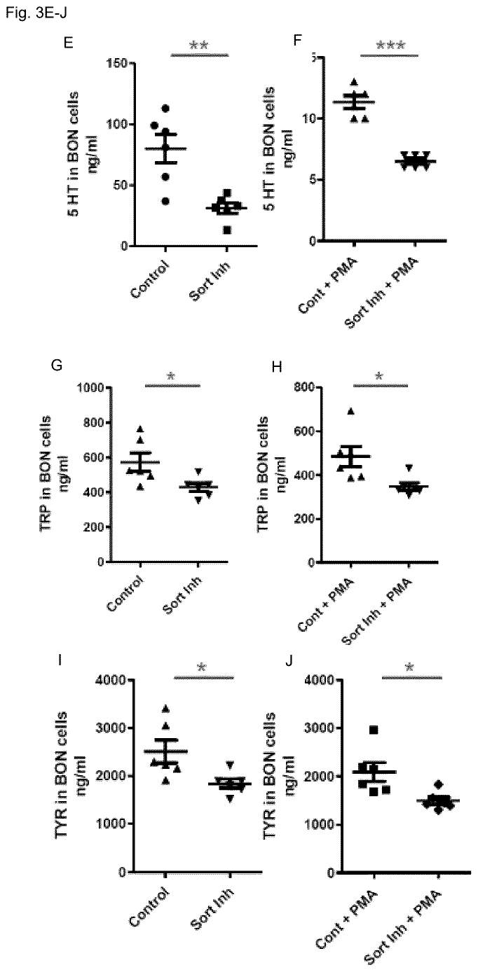

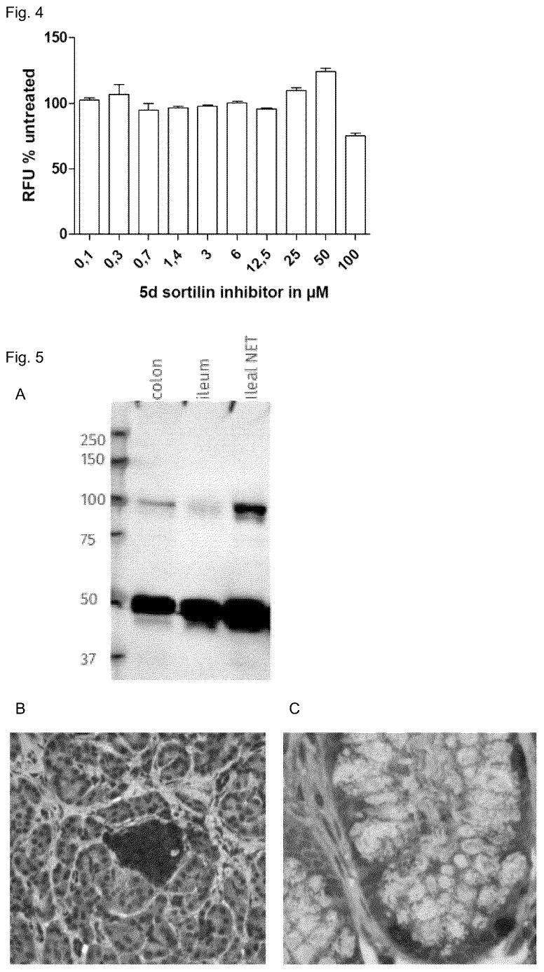

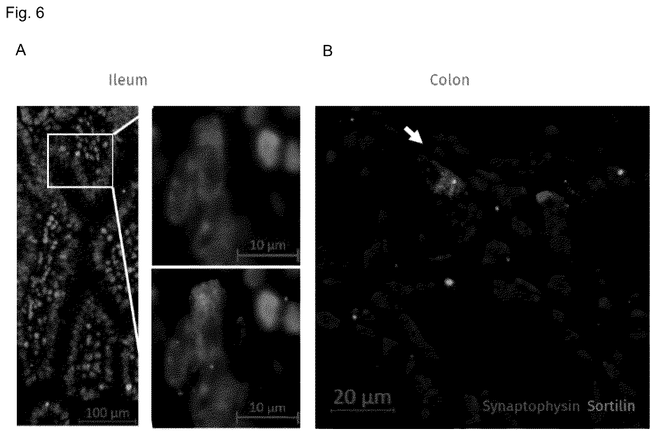

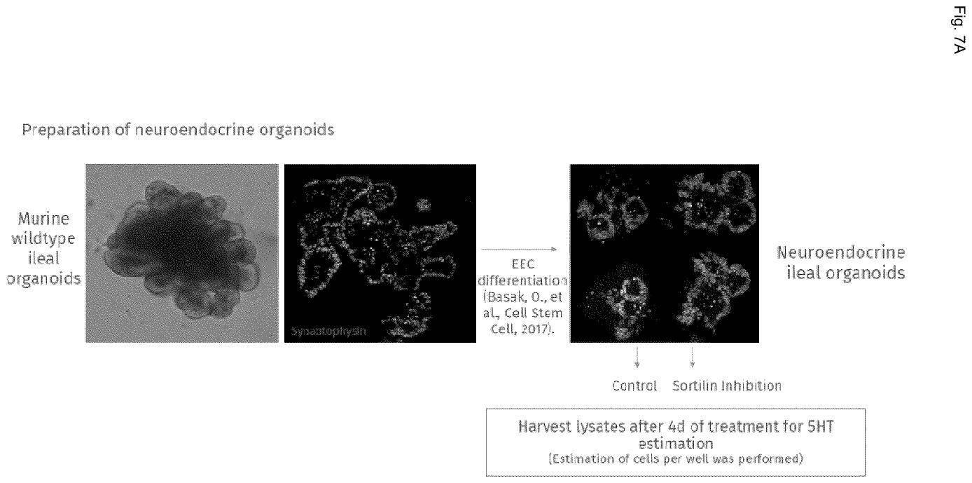

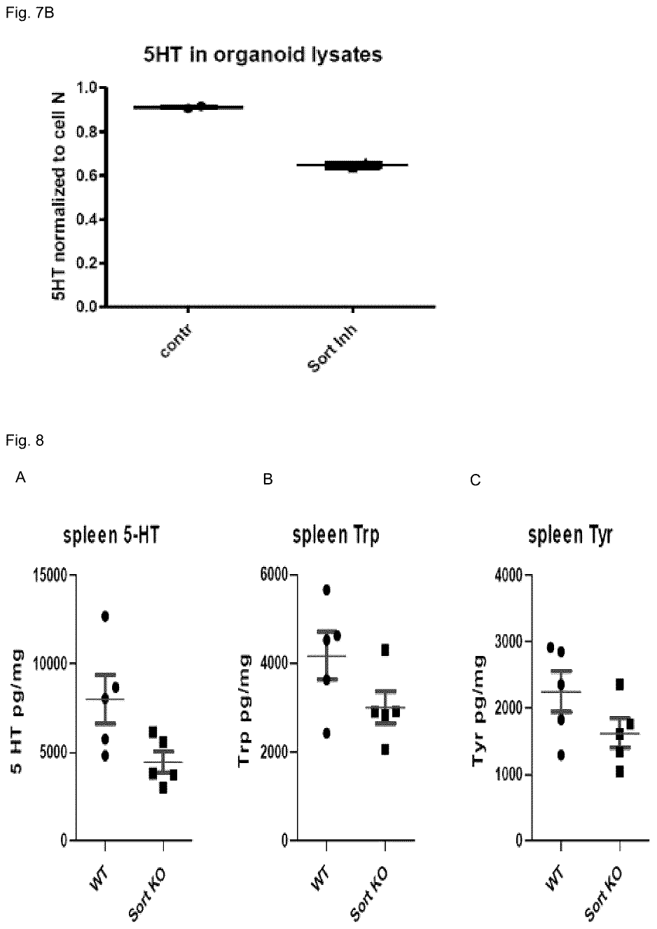

Next, we wanted to analyze whether sortilin has a functional role in hormone production or secretion of neuroendocrine tumors. We focused on one of the most relevant functional syndromes, i. e. the carcinoid syndrome which is caused due to the overproduction and secretion of serotonin (Clement et al., 2020). As a model we used the serotonin producing and sortilin expressing neuroendocrine BON cell culture (Kim et al., 2018; Samuel Tran et al., 2004). By Western blot analyses we could again confirm the expression of sortilin in BON cell cultures (FIG. 2). In addition, we treated BON cells 23 h with or without the sortilin inhibitor AF38469 (10 μM) in serotonin free medium. Afterwards we measured the serotonin (5HT) content in the cell culture supernatant by using high performance liquid chromatography (HPLC) (FIG. 3A-D). The cells were treated with fresh sortilin inhibitor for two more hours. Subsequently, the 5HT content in the cell lysates was measured by using HPLC as well (FIG. 3E-J). When compared to control conditions, the cellular content of 5HT was significantly lower after sortilin inhibition. As a result, the concentration of 5HT in the supernatants of treated cells was below the detection level. This finding indicates that targeting sortilin by its inhibitor represents a promising therapeutic approach for lowering the 5HT production and thereby treating the carcinoid syndrome.

To exclude the possibility of toxic effects of sortilin inhibition on the BON cells and thereby reducing 5HT production, we performed a cell proliferation assay using Alamar Blue. Under these experimental conditions, we could exclude any reduction in cell proliferation even after 5d of sortilin inhibition (FIG. 4).

It is widely accepted that the hormone production and secretion of NETs is similar to that of healthy neuroendocrine cells (Wiedenmann et al., 1998). In accordance with that, we could also detect sortilin in healthy tissue by Western blot analyses (FIG. 5A). Immunostaining revealed that the endocrine part of the pancreas (the pancreatic islet) was highly positive for sortilin (FIG. 5B). Staining the midgut for sortilin revealed scattered sortilin positive cells resembling neuroendocrine cells (FIG. 5C). Double immunostaining for synaptophysin and sortilin could validate this assumption (FIGS. 6A and B). This is in line with previous published studies, which could observe sortilin in neuroendocrine cells (Boggild et al., 2016).

We used murine intestinal organoids and enriched them with neuroendocrine cells as previously demonstrated (Kawasaki et al., 2018, Basak et al., 2017). Some of these organoids were treated additionally four days with the sortilin inhibitor AF38469 (10 μM). Subsequently, the lysates of these organoids were harvested, and serotonin was measured by HPLC (FIG. 7A). Organoids treated with the sortilin inhibitor showed a lower concentration of serotonin per cell (FIG. 7B). This result underlines the role of sortilin in hormone production in neuroendocrine cells and underpins sortilin as a promising therapeutic target.

In addition, we took advantage of a sortilin KO mouse model generated previously (Jansen et al, 2007). This mouse line was obtained by homologous recombination, thereby deleting 41 codons in exon 14 of the Sort1 gene. Mice homozygous for this deletion are viable and do not show apparent clinical alterations. Although not significant, we found that sortilin KO mice exhibited reduced 5HT levels in the spleen, which is indicative of a lower 5HT concentration in the blood (FIG. 8). As these mice lack completely sortilin starting already from the embryonic state on without an apparent phenotype, it is expected that compensatory mechanisms took place in order to attenuate the effect of the sortilin knock-out.

In summary we demonstrated a significant higher sortilin expression in functional active NETs than in non-functional NETs. Additionally, using NET cell culture, organoid and mouse models, we could show that a reduced sortilin function leads to a decrease in the serotonin concentration in various experimental settings. Therefore, sortilin represents a completely new target for the treatment of the functionality (e.g. carcinoid syndrome) of neuroendocrine tumors.

### REFERENCES

Ito T, Lee L, Jensen R T. Carcinoid-syndrome: recent advances, current status and controversies. Curr Opin Endocrinol Diabetes Obes. 2018 Feb. 25(1):22-35. doi: 10.1097/MED.0000000000000376. PMID: 29120923; PMCID: PMC5747542.
Miyakawa S, Sakuma H, Warude D, Asanuma S, Arimura N, Yoshihara T, Tavares D, Hata A, Ida K, Hori Y, Okuzono Y, Yamamoto S, Iida K, Shimizu H, Kondo S, Sato S. Anti-sortilin1 Antibody Up-Regulates Progranulin via Sortilin1 Down-Regulation. Front Neurosci. 2020 Dec. 15; 14:586107. doi: 10.3389/fnins.2020.586107. PMID: 33384578; PMCID: PMC7770147
Lee W C, Almeida S, Prudencio M, Caulfield T R, Zhang Y J, Tay W M, Bauer P O, Chew J, Sasaguri H, Jansen-West K R, Gendron T F, Stetler C T, Finch N, Mackenzie I R, Rademakers R, Gao F B, Petrucelli L. Targeted manipulation of the sortilin-progranulin axis rescues progranulin haploinsufficiency. Hum Mol Genet. 2014 Mar. 15; 23(6):1467-78. doi: 10.1093/hmg/ddt534. Epub 2013 Oct. 26. PMID: 24163244; PMCID: PMC3929086.
S, Canuel M, Zeng J, Morales C R. Inactivation of sortilin (a novel lysosomal sorting receptor) by dominant negative competition and RNA interference. Biol Proced Online. 2005; 7:17-25. doi: 10.1251/bpo101. Epub 2005 Jan. 25. PMID: 15682222; PMCID: PMC545973.
Gao F, Griffin N, Faulkner S, Li X, King S J, Jobling P, Denham J W, Jiang C C, Hondermarck H. The Membrane Protein Sortilin Can Be Targeted to Inhibit Pancreatic Cancer Cell Invasion. Am J Pathol. 2020 September; 190(9):1931-1942. doi: 10.1016/j.ajpath.2020.05.018. Epub 2020 Jun. 8. PMID: 32526166.
Rindi G, Wiedenmann B. Neuroendocrine neoplasia of the gastrointestinal tract revisited: towards precision medicine. Nature Reviews Endocrinology. 2020; 16(10):590-607.
Dasari A, Shen C, Halperin D, Zhao B, Zhou S, Xu Y, et al. Trends in the Incidence, Prevalence, and Survival Outcomes in Patients With Neuroendocrine Tumors in the United States. JAMA Oncology. 2017; 3(10):1335-42.
Halperin D M, Shen C, Dasari A, Xu Y, Chu Y, Zhou S, et al. Frequency of carcinoid syndrome at neuroendocrine tumour diagnosis: a population-based study. The Lancet Oncology. 2017; 18(4):525-34.
Joish V N, Shah S, Tierce J C, Patel D, McKee C, Lapuerta P, et al. Serotonin levels and 1-year mortality in patients with neuroendocrine tumors: a systematic review and meta-analysis. Future Oncology. 2019; 15(12):1397-406.
Malik A R, Willnow T E. VPS10P Domain Receptors: Sorting Out Brain Health and Disease. Trends Neurosci. 2020; 43(11):870-85.
Roselli S, Pundavela J, Demont Y, Faulkner S, Keene S, Attia J, et al. Sortilin is associated with breast cancer aggressiveness and contributes to tumor cell adhesion and invasion. Oncotarget. 2015; 6(12):10473-86.
Al-Akhrass H, Naves T, Vincent F, Magnaudeix A, Durand K, Bertin F, et al. Sortilin limits EGFR signaling by promoting its internalization in lung cancer. Nature Communications. 2017; 8(1):1182.
Gao F, Griffin N, Faulkner S, Rowe C W, Williams L, Roselli S, et al. The neurotrophic tyrosine kinase receptor TrkA and its ligand NGF are increased in squamous cell carcinomas of the lung. Sci Rep. 2018; 8(1):8135.
Kim J T, Napier D L, Weiss H L, Lee E Y, Townsend C M, Jr., Evers B M. Neurotensin Receptor 3/Sortilin Contributes to Tumorigenesis of Neuroendocrine Tumors Through Augmentation of Cell Adhesion and Migration. Neoplasia. 2018; 20(2):175-81. Schrøder T J, Christensen S, Lindberg S, Langgård M, David L, Maltas P J, Eskildsen J, Jacobsen J, Tagmose L, Simonsen K B, Biilmann Rønn L C, de Jong I E, Malik I J, Karlsson J J, Bundgaard C, Egebjerg J, Stavenhagen J B, Strandbygård D, Thirup S, Andersen J L, Uppalanchi S, Pervaram S, Kasturi S P, Eradi P, Sakumudi D R, Watson S P. The identification of AF38469: an orally bioavailable inhibitor of the VPS10P family sorting receptor Sortilin. Bioorg Med Chem Lett. 2014 Jan. 1; 24(1):177-80. doi: 10.1016/j.bmcl.2013.11.046. Epub 2013 Nov. 27. PMID: 24355129.
Lee W C, Almeida S, Prudencio M, Caulfield T R, Zhang Y J, Tay W M, Bauer P O, Chew J, Sasaguri H, Jansen-West K R, Gendron T F, Stetler C T, Finch N, Mackenzie I R, Rademakers R, Gao F B, Petrucelli L. Targeted manipulation of the sortilin-progranulin axis rescues progranulin haploinsufficiency. Hum Mol Genet. 2014 Mar. 15; 23(6):1467-78. doi: 10.1093/hmg/ddt534. Epub 2013 Oct. 26. PMID: 24163244; PMCID: PMC3929086.
WO2021186054A1
Stachel et al., 2020, Identification of potent inhibitors of the sortilin-progranulin interaction. Bioorg Med Chem Lett.
Anand et al., 2023, Discovery of novel small molecule inhibitors targeting progranulin-sortilin: A virtual high throughput screening approach.
WO2009132656A2
WO2020014617A1
WO2016/164637A1
WO2022/120352A1
Felix F. Adams, Dirk Heckl, Thomas Hoffmann, Steven R. Talbot, Arnold Kloos, Felicitas Thol, Michael Heuser, Johannes Zuber, Axel Schambach, Adrian Schwarzer,
An optimized lentiviral vector system for conditional RNAi and efficient cloning of microRNA embedded short hairpin RNA libraries, Biomaterials, Volume 139, 2017, Pages 102-115, ISSN 0142-9612, https://doi.org/10.1016/j.biomaterials.2017.05.032.
Roselli S., Pundavela J., Demont Y., Faulkner S., Keene S., Attia J., Jiang C. Chen, Zhang X. Dong, Walker M. M., Hondermarck H. Sortilin is associated with breast cancer aggressiveness and contributes to tumor cell adhesion and invasion. Oncotarget. 2015; 6: 10473-10486.
Lefrancois, S., Canuel, M., Zeng, J. et al. Inactivation of sortilin (a novel lysosomal sorting receptor) by dominant negative competition and RNA interference. BioL. Proced. Online 7, 17-25 (2005). https://doi.org/10.1251/bpo101.
Uchiyama K, Tomita M, Yano M, Chida J, Hara H, Das N R, Nykjaer A, Sakaguchi S. Prions amplify through degradation of the VPS10P sorting receptor sortilin. PLoS Pathog. 2017 Jun. 30; 13(6):e1006470. doi: 10.1371/journal.ppat.1006470. PMID: 28665987; PMCID: PMC5509376.
Gao F, Griffin N, Faulkner S, Li X, King S J, Jobling P, Denham J W, Jiang C C, Hondermarck H. The Membrane Protein Sortilin Can Be Targeted to Inhibit Pancreatic Cancer Cell Invasion. Am J Pathol. 2020 September; 190(9):1931-1942. doi: 10.1016/j.ajpath.2020.05.018. Epub 2020 Jun. 8. PMID: 32526166.
Selvam C, Mutisya D, Prakash S, Ranganna K, Thilagavathi R. Therapeutic potential of chemically modified siRNA: Recent trends. Chem Biol Drug Des. 2017 November; 90(5):665-678. doi: 10.1111/cbdd.12993. Epub 2017 May 16. PMID: 28378934; PMCID: PMC5935465. Clement D, Ramage J, Srirajaskanthan R. Update on Pathophysiology, Treatment, and Complications of Carcinoid Syndrome. Journal of Oncology. 2020; 2020:8341426.
SAMUEL TRAN V, MARION-AUDIBERT A-M, KARATEKIN E, HUET S, CRIBIER S, GUILLAUMIE K, et al. Serotonin Secretion by Human Carcinoid BON Cells. Annals of the New York Academy of Sciences. 2004; 1014(1):179-88.
Contractor T, Clausen R, Harris G R, Rosenfeld J A, Carpizo D R, Tang L, et al. IGF2 drives formation of ileal neuroendocrine tumors in patients and mice. Endocrine-Related Cancer. 2020; 27(3):175-86.
Kawasaki K, Fujii M, Sato T. Gastroenteropancreatic neuroendocrine neoplasms: genes, therapies and models. Disease Models & Mechanisms. 2018; 11(2).
Kawasaki K, Toshimitsu K, Matano M, Fujita M, Fujii M, Togasaki K, et al. An Organoid Biobank of Neuroendocrine Neoplasms Enables Genotype-Phenotype Mapping. Cell. 2020; 183(5):1420-35.e21.
Wiedenmann B, John M, Ahnert-Hilger G, Riecken E-O. Molecular and cell biological aspects of neuroendocrine tumors of the gastroenteropancreatic system. Journal of Molecular Medicine. 1998; 76(9):637-47.
Boggild S, Molgaard S, Glerup S, Nyengaard J R. Spatiotemporal patterns of sortilin and SorCS2 localization during organ development. BMC Cell Biol. 2016; 17:8-.
Basak O, Beumer J, Wiebrands K, Seno H, van Oudenaarden A, Clevers H. Induced Quiescence of Lgr5+ Stem Cells in Intestinal Organoids Enables Differentiation of Hormone-Producing Enteroendocrine Cells. Cell Stem Cell. 2017; 20(2):177-90.e4.
Jansen P, Giehl K, Nyengaard J R, Teng K, Lioubinski O, Sjoegaard S S, et al. Roles for the pro-neurotrophin receptor sortilin in neuronal development, aging and brain injury. Nat Neurosci. 2007; 10(11):1449-57.

1-14. (canceled)

15. A method of treating a condition associated with a release of an endocrine and/or a paracrine factor by a functional neuroendocrine tumor (NET) in a subject, wherein the method comprises administering to the subject a therapeutically effective amount of a sortilin inhibitor.

16. The method of claim 15, wherein the endocrine and/or paracrine factor is selected from the group consisting of serotonin, histamine, bradykinin, tachykinin (substance P), prostaglandin, kallikrein, adrenaline, gastrin, insulin, glucagon, somatostatin, corticotropin-releasing hormone, adrenocorticotropin, somatoliberin, somatotropin, parathyroid hormone-related protein, calcitonin, calcitonin gene-related peptide, vasoactive intestinal polypeptide, chromogranin A, katacalcin, neuropeptide Y, bombesin/gastrin releasing peptide (GRP), alpha-human chorionic gonadotropin (aHCG), thyroid stimulating hormone-like peptide, cholecystokinin, adrenomedullin and vascular endothelial growth factor (VEGF) and any combination thereof.

17. The method of claim 16, wherein the endocrine factor is serotonin.

18. The method of claim 15, wherein the condition is selected from the group comprising carcinoid syndrome, carcinoid heart disease, carcinoid crisis, niacin deficiency, pellagra, dermatitis, dementia, diarrhea, acromegaly, Cushing's syndrome; low blood sugar, skin rashes, disturbed sugar metabolism, Verner-Morrison syndrome and Zollinger-Ellison syndrome.

19. The method of claim 18, wherein the condition is carcinoid syndrome.

20. The method of claim 15, wherein the sortilin inhibitor is selected from the group comprising a small molecule inhibitor having a molecular weight of 800 daltons or less, an anti-sortilin antibody, an inhibitory nucleic acid, a proteolysis-targeting chimera and a peptide inhibitor.

21. The method of claim 15, wherein the sortilin inhibitor is a small molecule inhibitor having a molecular weight of 800 daltons or less selected from the group comprising 2-[[(6-methyl-2-pyridinyl)amino]carbonyl]-5-(trifluoromethyl)-benzoic acid, 1-[2-(2-tert-butyl-5-methylphenoxy)-ethyl]-3-methylpiperidine, (2S)-2-{[(7-hydroxy-4-methyl-2-oxo-2H-chromen-8-yl)methyl]amino}-4-methylpentanoic acid, 2-[(6-methylpyridin-2-yl)carbamoyl]benzoic acid, 2-methyl-6-[(6-methylpyridin-2-yl)carbamoyl]benzoic acid, 5-bromo-2-[(6-methylpyridin-2-yl)carbamoyl]benzoic acid, 5-chloro-2-[(6-methylpyridin-2-yl)carbamoyl]benzoic acid, 5-methyl-2-[(6-methylpyridin-2-yl)carbamoyl]benzoic acid, 2-[(6-methylpyridin-2-yl)carbamoyl]-5-(propan-2-yl)benzoic acid, 2-[(6-methylpyridin-2-yl)carbamoyl]-5-(trifluoromethyl)benzoic acid, 2-[(6-methylpyridin-2-yl)carbamoyl]-5-nitrobenzoic acid, 4-[(6-methylpyridin-2-yl)carbamoyl]-[1,1′-biphenyl]-3-carboxylic acid, 4-bromo-2-[(6-methylpyridin-2-yl)carbamoyl]benzoic acid, 4-chloro-2-[(6-methylpyridin-2-yl)carbamoyl]benzoic acid, 4-methyl-2-[(6-methylpyridin-2-yl)carbamoyl]benzoic acid, 2-[(6-methylpyridin-2-yl)carbamoyl]-4-(trifluoromethyl)benzoic acid, 4,5-dichloro-2-[(6-methylpyridin-2-yl)carbamoyl]benzoic acid, 4,5-dimethyl-2-[(6-methylpyridin-2-yl)carbamoyl]benzoic acid; 3-methyl-2-[(6-methylpyridin-2-yl)carbamoyl]benzoic acid, 5-bromo-2-[(butan-2-yl)carbamoyl]benzoic acid, 5-bromo-2-[(propan-2-yl)carbamoyl]benzoic acid, 5-bromo-2-(phenylcarbamoyl)benzoic acid, 5-bromo-2-[(3-methylphenyl)carbamoyl]benzoic acid, 5-bromo-2-[(pyridin-2-yl)carbamoyl]benzoic acid, 5-bromo-2-[(6-chloropyridin-2-yl)carbamoyl]benzoic acid, 5-bromo-2-[(4-methylpyridin-2-yl)carbamoyl]benzoic acid, 5-bromo-2-[(3-methylpyridin-2-yl)carbamoyl]benzoic acid, 5-bromo-2-[(6-methylpyridin-3-yl)carbamoyl]benzoic acid, 5-bromo-2-[(2-methylpyridin-4-yl)carbamoyl]benzoic acid, 5-bromo-2-[(2-methylpyrimidin-4-yl)carbamoyl]benzoic acid, 2-[(6-methoxypyridin-2-yl)carbamoyl]-5-(trifluoromethyl)benzoic acid, 2-[(5,6-dimethylpyridin-2-yl)carbamoyl]-5-(trifluoromethyl)benzoic acid, 2-[(5,6,7,8-tetrahydroquinolin-2-yl)carbamoyl]-5-(trifluoromethyl)benzoic acid, or a pharmaceutically acceptable salt, solvate, hydrate, tautomer, optical isomer, N-oxide, and/or prodrug thereof.

22. The method of claim 15, wherein the sortilin inhibitor is an anti-sortilin antibody.

23. The method of claim 22, wherein the anti-sortilin antibody is selected from the group consisting of
an anti-sortilin antibody having a heavy chain with an amino acid sequence of SEQ ID NO: 1 and a light chain with an amino acid sequence of SEQ ID NO: 2 and
an anti-sortilin antibody comprising a light chain variable domain and a heavy chain variable domain, wherein the heavy chain variable domain comprises an HVR—H1 comprising the amino acid sequence of SEQ ID NO:3, an HVR-H2 comprising the amino acid sequence of SEQ ID NO: 4, and an HVR-H3 comprising the amino acid sequence of SEQ ID NO:5, and the light chain variable domain comprises an HVR-L1 comprising the amino acid sequence of SEQ ID NO:6, an HVR-L2 comprising the amino acid sequence of SEQ ID NO: 7, and an HVR-L3 comprising the amino acid sequence of SEQ ID NO:8.

24. The method of claim 15, wherein the sortilin inhibitor is an inhibitory nucleic acid, wherein the inhibitory nucleic acid is
a) an antisense oligonucleotide; or
b) an inhibitory RNA selected from the group comprising an siRNA, an shRNA or a miRNA; or
an aptamer selected from the group comprising a DNA aptamer, an RNA aptamer, an XNA aptamer and an L-ribonucleic acid aptamer.

25. The method of claim 24, wherein the inhibitory nucleic acid is an siRNA comprising a nucleic acid sequence of any of SEQ ID NO: 9-14.

26. The method of claim 15, wherein the sortilin inhibitor is a peptide inhibitor.

27. The method of claim 15, wherein the sortilin inhibitor is provided in a pharmaceutical composition.

28. The method of claim 27, wherein the pharmaceutical composition comprises a vector, wherein,
in case the sortilin inhibitor is a small molecule inhibitor having a molecular weight of 800 daltons or less, an anti-sortilin antibody, an in-vitro produced inhibitory nucleic acid, a proteolysis-targeting chimera or a peptide-based inhibitor, the vector comprises the sortilin inhibitor as a cargo and is selected from the group comprising a liposomal or an exosomal carrier, a lipid nanoparticle, a bacterial minicell, a polymeric micro- or nanoparticle and a nanocarrier comprising a hyperbranched dendritic polyglycerol polymer; or
in case the sortilin inhibitor is an inhibitory nucleic acid, the vector is an expression vector selected from the group comprising minicircles, plasmids, cosmids, phages, viruses or artificial chromosomes encoding the sortilin inhibitor.

29. The method of claim 15, wherein the functional NET is selected from the group comprising a small intestine NET, an appendix NET, a colon NET, a lung NET, a pancreas NET, pheochromocytoma and a paraganglioma.

30. The method of claim 15, wherein the condition associated with the release of an endocrine and/or a paracrine factor by a functional neuroendocrine tumor is carcinoid syndrome, the factor is serotonine and the sortilin inhibitor is 2-[[(6-methyl-2-pyridinyl)amino]carbonyl]-5-(trifluoromethyl)-benzoic acid, an anti-sortilin antibody having a heavy chain with an amino acid sequence of SEQ ID NO: 1 and a light chain with an amino acid sequence of SEQ ID NO: 2, or an anti-sortilin antibody comprising a light chain variable domain and a heavy chain variable domain, wherein the heavy chain variable domain comprises an HVR—H1 comprising the amino acid sequence of SEQ ID NO:3, an HVR-H2 comprising the amino acid sequence of SEQ ID NO: 4, and an HVR-H3 comprising the amino acid sequence of SEQ ID NO:5, and the light chain variable domain comprises an HVR-L1 comprising the amino acid sequence of SEQ ID NO:6, an HVR-L2 comprising the amino acid sequence of SEQ ID NO: 7, and an HVR-L3 comprising the amino acid sequence of SEQ ID NO:8.
